SEQUENCE LISTING (19 sequences)

SEQ ID NO: 1 - DNA, 40 bp, Artificial Sequence
  Pci I deletion primer 1
gctggccttttgctcagatggtctttcctgcgttatcccc

SEQ ID NO: 2 - DNA, 40 bp, Artificial Sequence
  Pci I deletion primer 2
ggggataacgcaggaaagaccatgtgagcaaaaggccagc

SEQ ID NO: 3 - DNA, 48 bp, Artificial Sequence
  NCAM primer 1
gctactaagcttgccgccagcatggtgcaaactaaggatctcatctgg

SEQ ID NO: 4 - DNA, 54 bp, Artificial Sequence
  NCAM primer 2
gctgatctccccctggctgggaaacatgtccacctgcagagaaactgcagttcc

SEQ ID NO: 5 - DNA, 44 bp, Artificial Sequence
  NCAM primer 3
gccgtagtctcgagtcctgtagatgtcctgaacacaaaatgagc

SEQ ID NO: 6 - DNA, 61 bp, Artificial Sequence
  Ig5-FN1 primer 1
cctattaacatgtcatctggagcagcggccgcatatgccccaaagctacagggccctgtgg

SEQ ID NO: 7 - DNA, 41 bp, Artificial Sequence
  Ig5-FN1 primer 2
cgtagtctcgagtccctgcttgatcaggttcactttaatag

SEQ ID NO: 8 - DNA, 62 bp, Artificial Sequence
  FN1 primer 1
cctattaacatgtcatctggagcagcggccgcattcatccttgttcaagcagacaccccctc

SEQ ID NO: 9 - DNA, 41 bp, Artificial Sequence; identical to SEQ ID NO: 7
  FN1 primer 2

SEQ ID NO: 10 - DNA, 27 bp, Artificial Sequence
  Ig5-FN1 fusion primer 1
tttgggctcgagtatgccccaaagcta

SEQ ID NO: 11 - DNA, 27 bp, Artificial Sequence
  Ig5-FN1 fusion primer 2
tttgggctcgagtccctgcttgatcag

SEQ ID NO: 12 - DNA, 27 bp, Artificial Sequence
  Ig5 fusion primer 1
tttgggactgattatgccccaaagcta

SEQ ID NO: 13 - DNA, 30 bp, Artificial Sequence
  Ig5 fusion primer 2
tttgggactgattgcttgaacaaggatgaa

SEQ ID NO: 14 - DNA, 24 bp, Artificial Sequence
  Cla I primer 1
atccttgttactgatgacaccccc

SEQ ID NO: 15 - DNA, 24 bp, Artificial Sequence
  Cla I primer 2
gggggtgtcatcagtaacaaggat

SEQ ID NO: 16 - DNA, 33 bp, Artificial Sequence
  scFv glycosylation primer 1
tattactgccagaactgtactagttacccactc

SEQ ID NO: 17 - DNA, 33 bp, Artificial Sequence
  scFv glycosylation primer 2
gagtgggtaactagtacagttctggcagtaata

SEQ ID NO: 18 - DNA, 1542 bp, Artificial Sequence
  NCAM fusion
atgctgcaaactaaggatctcatctggactttgtttttcctgggaactgcagtttctctgcaggtggacatggcccaggtgaaactgcagcagtctggggcagaacttgtgaggtcagggacctcagtcaagttgtcctgcacagcttctggcttcaacattaaagactcctatatgcactggttgaggcaggggcctgaacagggcctggagtggattggatggattgatcctgagaatggtgatactgaatatgccccgaagttccagggcaaggccacttttactacagacacatcctccaacacagcctacctgcagctcagcagcctgacatctgaggacactgccgtctattattgtaatgaggggactccgactgggccgtactactttgactactggggccaagggaccacggtcaccgtctcctcaggtggaggcggttcaggcggaggtggctctggcggtggcggatcagaaaatgtgctcacccagtctccagcaatcatgtctgcatctccaggggagaaggtcaccataacctgcagtgccagctcaagtgtaagttacatgcactggttccagcagaagccaggcacttctcccaaactctggatttatagcacatccaacctggcttctggagtccctgctcgcttcagtggcagtggatctgggacctcttactctctcacaatcagccgaatggaggctgaagatgctgccacttattactgccagcaaaggagtagttacccactcacgttcggtgctggcaccaagctggagctgaaacgggcggccgcatatgccccaaagctacagggccctgtggctgtgtacacttgggaggggaaccaggtgaacatcacctgcgaggtatttgcctatcccagtgccacgatctcatggtttcgggatggccagctgctgccaagctccaattacagcaatatcaagatctacaacaccccctctgccagctatctggaggtgaccccagactctgagaatgattttgggaactacaactgtactgcagtgaaccgcattgggcaggagtccttggaattcatccttgttcaagcagacaccccctcttcaccatccatcgaccaggtggagccatactccagcacagcccaggtgcagtttgatgaaccagaggccacaggtggggtgcccatcctcaaatacaaagctgagtggagagcagtgggtgaagaagtatggcattccaagtggtatgatgccaaggaagccagcatggagggcatcgtcaccatcgtgggcctgaagcccgaaacaacgtacgccgtaaggctggcggcgctcaatggcaaagggctgggtgagatcagcgcggcctccgagttcaagacgcagccagtccaaggggaacccagtgcacctaagctcgaagggcagatgggagaggatggaaactctattaaagtgaacctgatcaagcagggactcgagcatcatcatcatcatcatgggtccgaacaaaaactcatctcagaagaggatctgtga

SEQ ID NO: 19 - protein, 513 aa, Artificial Sequence
  NCAM fusion
MLQTKDLIWTLFFLGTAVSLQVDMAQVKLQQSGAELVRSGTSVKLSCTASGFNIKDSYMHWLRQGPEQGLEWIGWIDPENGDTEYAPKFQGKATFTTDTSSNTAYLQLSSLTSEDTAVYYCNEGTPTGPYYFDYWGQGTTVTVSSGGGGSGGGGSGGGGSENVLTQSPAIMSASPGEKVTITCSASSSVSYMHWFQQKPGTSPKLWIYSTSNLASGVPARFSGSGSGTSYSLTISRMEAEDAATYYCQQRSSYPLTFGAGTKLELKRAAAYAPKLQGPVAVYTWEGNQVNITCEVFAYPSATISWFRDGQLLPSSNYSNIKIYNTPSASYLEVTPDSENDFGNYNCTAVNRIGQESLEFILVQADTPSSPSIDQVEPYSSTAQVQFDEPEATGGVPILKYKAEWRAVGEEVWHSKWYDAKEASMEGIVTIVGLKPETTYAVRLAALNGKGLGEISAASEFKTQPVQGEPSAPKLEGQMGEDGNSIKVNLIKQGLEHHHHHHGSENKLISEEDL

(19) United States
(12) Patent Application Publication
(10) Pub. No.: US 2010/0069321 A1
(43) Pub. Date: Mar. 18, 2010

(54) Biological Materials and Uses Thereof

(75) Inventors: Mahendra Deonarain, London (GB); Agamemnon Epenetos, London (GB); Anthony Constantinou, Birmingham (GB)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: IMPERIAL INNOVATIONS LIMITED, London (GB)

(21) Appl. No.: 12/439,423

(22) PCT Filed: Aug. 31, 2007
(86) PCT No.: PCT/GB07/03284

§ 371 Date: Oct. 23, 2009

(30) Foreign Application Priority Data
Sep. 1, 2006 (GB) 0617241.5

(51) Int. Cl. C12P 19/04; C07H 1/00; C07H 21/04; C12N 15/63; C12N 5/10; A61K 31/715

(52) U.S. Cl. 514/54; 435/101; 536/123.1; 536/23.1; 435/320.1; 435/325

(57) ABSTRACT

The invention relates to the modification of proteins to improve their, biochemical, immunological or biophysical properties, in turn leading to such proteins having increased diagnostic, biotechnological or therapeutic benefit. In particular the invention relates to polysialylation of proteins or conjugates of proteins. There is also provided, nucleotide sequences and expression vectors encoding, host cells expressing, compositions comprising and uses of the polysialylated molecules of the invention.

The invention relates to the recombinant modification of proteins to improve their biochemical, immunological or biophysical properties, thereby producing proteins having increased diagnostic, biotechnological or therapeutic benefit. In particular the invention relates to polysialylation of proteins or conjugates of proteins.

Drugs comprising active proteins such as antibodies, insulin, interferon and erythropoietin have been used therapeutically for many years. Moreover, antibodies represent the largest class of biotechnological protein drugs being developed. Advances in genomics, proteomics and pharmacogenomics are increasing the impact and relevance of these drugs: new and more specific targets and a better understanding of the biological responses are helping to make future generations of protein-based drugs more effective and even tailor-made for specific groups of individuals [1].

However, protein-based drugs are often compromised by limitations due to their complex molecular structure [2,3,4]. This includes rapid elimination from the blood before effective concentrations are reached, rapid clearance leading to a short therapeutic window, proteolytic degradation, uptake by cells of the reticulo-endothelial system, excretion via the renal route and immuno-complex formation. The major factors which contribute to these pharmacologic, pharmacodynamic and pharmacokinetic limitations are protein size [5], glycosylation [6], stability [7] and immunogenicity [8].

Antibodies represent a characteristic molecule that can be used as a protein based drug. Antibodies have naturally evolved to act as the first line of defense in the mammalian immune system. They are complex glycoproteins which have excellent target specificity and tremendous diversity resulting from programmed gene shuffling and targeted mutagenesis [45]. This diversity is such that antibodies can bind to practically any target molecule which is usually (but not always) proteinaceous in nature.

It is now possible to mimic antibody selection and production in vitro, selecting for recombinant human antibodies against a desired target [46]. The most popular in vitro selection technique is antibody phage display, where antibodies are displayed and manipulated on the surface of viruses.

Taking antibodies as an example of a ligand that is capable of binding a specific target, antibodies can bind with a variable degree of specificity to target cells expressing the appropriate receptor or a soluble target

The affinity of an antibody is a measure of how well an antibody binds to the target (antigen). It is usually described by an equilibrium dissociation constant (Kd, units M) or equilibrium association constant (Ka, units M⁻¹). The affinity constant is a function of the two kinetic constants kₒₙ and k_{off}. The rate of association is dependent on the kₒₙ rate constant (units M⁻¹ s⁻¹) and the rate of dissociation is dependent on the k_{off} rate constant (units s⁻¹). Technology exists to select and manipulate antibodies which have the desired kinetic binding properties [47]. For antibodies that need to be internalised to deliver a cytotoxic drug, the association rate is more important as the dissociation rate does not apply if the antibody is taken into the cell [48]. For example, for antibodies which neutralise cytokines or toxins [49], a rapid association rate may be more beneficial.

Issues of binding affinity apply equally to all anti-ligand/ligand pairs and it is generally accepted that affinity is related to biological response. In medicine, increased affinity and more specifically targeted binding can lead to lower doses and subsequently lower costs.

As with all biological molecules, the size of the antibody affects its pharmacokinetics in vivo [5]. Larger molecules persist longer in the circulation due to slow clearance (large glycoproteins are cleared through specific uptake by the liver). For whole antibodies (approximate molecular weight of 150 KDa) which recognise a cancer cell antigen in an experimental mouse model system, 30-40% can be taken up by the tumour, but because they persist longer in the circulation, it takes 1-2 days for a tumour:blood ratio of more than one to be reached. Typical tumour: blood ratios are 5-10 by about day 3. With smaller fragments of antibodies, which have been produced by in vitro techniques and recombinant DNA technology, the clearance from the circulation is faster (molecules smaller than about 50 KDa are excreted through the kidneys).

Single-chain Fvs (about 30 KDa) are artificial binding molecules derived from whole antibodies, but contain the minimal part required to recognise antigen [51]). Again, it has been shown in mouse model systems, scFvs can deliver 1-2% of the injected dose, but with tumour: blood ratios better than 20:1.

There has been much research into targetable therapeutic drugs where novel effector functions have been linked to antibodies or other targeting ligands. Some of these need to be internalised to successfully deliver a toxic agent.

Immunotoxins have shown a number of problems such as causing immune reactions and liver/kidney toxicity. There have been developments with new ‘humanised’ immunotoxins based on enzymes such as ribonuclease [55] and deoxyribonuclease [56]. These potentially have lower side effects making them more tolerable, but they still do not have a bystander killing effect.

Chemotherapy drugs tend to be much less active when linked to proteins [48] as they do not get released effectively, thus requiring selectively cleavable chemical linkers. Radioimmunotherapy [32] tends to irradiate other tissues en route to the tumour, causing bone marrow and liver toxicity. Photosensitising (PS) drugs may also be linked to proteins as the cytotoxic elements are the singlet oxygen and other reactive oxygen species generated from them and not the PS drugs themselves [57].

Although antibodies are the first choice when it comes to considering ligands for targeting or detection, there exist many alternative ligands, some of which have been exploited through phage (or other) display/selection techniques. These include but are not limited to natural ligands for receptors (e.g. interleukin-6 (IL-6) [58] and tissue necrosis factor (TNF) [59], peptides (e.g. neuropeptides [60]) immunoglobulin-like domains (such as fibronectin (FN) domains [61], single immunoglobulin domains [62]), anticalins [63] and ankyrin repeats [64]. Many of these can be engineered and optimised to improve their biological and therapeutic properties.

There are many situations where the half-life of an active protein e.g. an antibody, would need to be increased or modulated in order to be an effective drug. For example, for antibodies removing the Fc-portion will reduce non-target tissue cross-reactivity and affect clearance, increase expression yields and allow more predictable and controlled pharmacokinetics.

One of the most important areas of improvement for antibodies is that of immunotoxicotherapy, where antibodies neutralize blood-borne factors such as toxins, cytokines, clotting receptors and narcotics in order to inhibit their effects or alter their tissue distribution. A prime example is that of the newly-licensed antibody Avastin™ which neutralizes vascular endothelial growth factor (VEGF) thereby preventing vascularization and growth of colorectal cancer [71,72]. Increased longevity of Avastin™ without the problems with Fc-mediated cross-reaction would be beneficial. Table 1 lists more examples of proteins which could be improved for therapy by modulating their serum half-lives.

TABLE 1
Proteins which can be improved by serum half-life modulation

| Possible proteins for improvement | Refs |
|---|---|
| Anti-Vascular endothelial growth factor (VEGF) antibody | 71, 72 |
| Anti-Tissue necrosis factor alpha (TNFa) antibody | 73. 74 |
| Anti- Anti-GPIIb/IIa blood coagulation factors antibody | 75 |
| Anti-Human immunodeficiency virus (HIV) antibody | 76 |
| Anti-Interleukin-6 antibody | 77 |
| Anti-Botulinum toxin antibody | 78 |
| Anti-Anthrax toxin antibody | 79 |
| Insulin | 80 |
| Erythropoietin | 81 |
| Anti-Interferon antibody | 82 |
| Anti-Narcotics antibodies | 83 |

A variety of strategies have been employed in the fields of protein chemistry and engineering in order to alleviate some of the limitations of therapeutic proteins, for example: encapsulation into liposomes to shield proteins from the immune system [9], site-directed mutagenesis to alter biophysical properties thereby improving stability [10] or conjugation of polymers to the active protein to alter the pharmacokinetic profile [4,11,24-31].

Of all the approaches to improve protein pharmacokinetics, polymer conjugation using poly-ethylene glycol (PEG), a process also known as PEGylation, has been one of the most successful and widely used [4,11-15].

PEG is a neutral polymer that can bind water molecules forming a ‘watery cloud’ around the compound e.g. drug, it is attached to. This gives the PEG-compound conjugate a larger hydrodynamic volume compared to its true molecular weight, For example a 30 KDa protein plus a 40 KDa PEG has a combined mass of 70 KDa but an apparent size of 360 KDa (as measured by size exclusion chromatography [13]). This will affect its pharmacokinetics and pharmacodynamics in the body. In addition to PEG causing changes in size, PEGylation also causes the protein surface charge to be modified and biological epitopes are commonly shielded from potential immune responses.

A number of PEGylated proteins have been approved for clinical use such as Oncaspar™ (PEG-asparginase) for the treatment of lymphoblastic leukaemic [11,16] and PEGasys™ (PEG-interferon-α 2a) for the treatment of chronic hepatitis C infections [12,17].

However, PEG is a synthetic polymer and there have been some concerns as to the metabolism and immunogenicity of PEG conjugates. For example, it has been shown that cells of the reticuloendothelial system (RES) and liver can take up small amounts of PEG conjugates and although the metabolism of PEG is as yet unclear, it is thought that PEG accumulates in lysosomes which could lead to toxicity [18]. More recently it has been shown that repeated administration of PEG conjugates can result in the production of anti-PEG antibodies [19].

Molecules which are inconspicuous to the innate and adaptive immune systems are more likely to survive for prolonged periods in the circulation. Neurotropic bacteria such as Neisseria meningitidis and some E. coli strains naturally synthesise a polysaccharide capsule consisting of polysialylic acid (PSA) a polymer of sialic acid [20]. Bacterial PSA is non-immunogenic in humans [21] because a PSA polymer is also found in humans, but only on a small number of proteins.

Polysialic acid is a developmentally regulated, anti-adhesive glycan which terminates N- or O-linked oligosaccharides found on a small group of glycoproteins. In mammals, it is usually found as a linear homopolymer of 50-100 units of α2,8-linked 5-N-acetylneuramic acid [34].

In humans, polysialylation is rare due to only a small number of proteins having sites which may be polysialylated. These naturally polysialylated human proteins include the alpha-subunit of the voltage-dependent sodium channel [35], a form of the CD36 scavenger receptor [36] and the two polysialyltransferase (PST and STX) enzymes [37] which autopolysialylate their own N-glycans as well as their substrate and NCAM (neural cell adhesion molecule) which is the most abundant polysialylated protein. PSA found on NCAM (neural cell adhesion molecule) plays an anti-adhesive role in brain development and tumour metastases [22].

Bacterial PSA is chemically and immunologically identical to human PSA and has been under development as an alternative to PEG for the purposes of improving immunogenicity, stability, pharmacokinetics and pharmacodynamics of therapeutic molecules [24-31]. Its highly hydrophilic nature results in similar hydration properties to PEG giving it a high apparent molecular weight.

PSA chains have been attached, using linking chemicals, to small active proteins [24], liposomes and non-antibody proteins [25-27] that do not naturally bear PSA chains. The commonest site of attachment to proteins is via surface lysine amino groups using N-hydroxy succinimide-ester chemistry or onto cysteine thiol groups via maleimido-derivatised PSA polymers.

Chemical polysialylation of insulin [25], asparaginase [26] and catalase [27] has resulted in improved stability and pharmacokinetics of each whilst preserving their normal function.

Recombinant antibody fragments have also been polysialylated leading to a range of improved properties in vivo [28,29]. Fab fragments have been chemically polysialylated with a range of different lengths and ratios of linear PSA chains [28] and for example chemical polysialylation of an anti-placental alkaline phosphatase Fab fragment resulted in a 4-fold decrease in blood clearance (t1/2β) with a corresponding 3-fold increase in tumour uptake compared to the unmodified Fab [28].

There remains the need to provide further improved therapeutic proteins, for example antibodies, in order to further improve immunogenicity, stability, pharmacokinetics and pharmacodynamics.

In a first aspect of the invention there is provided a method of polysialylation comprising the steps of:
(i) providing a molecule comprising a first protein or domain thereof associated with a second protein or domain thereof containing a natural polysialylation site;
(ii) exposing the molecule of step (i) to a polysialyltransferase enzyme so as to produce a polysialylated molecule wherein the polysialylation is a sugar chain N-linked onto an asparagine amino acid.

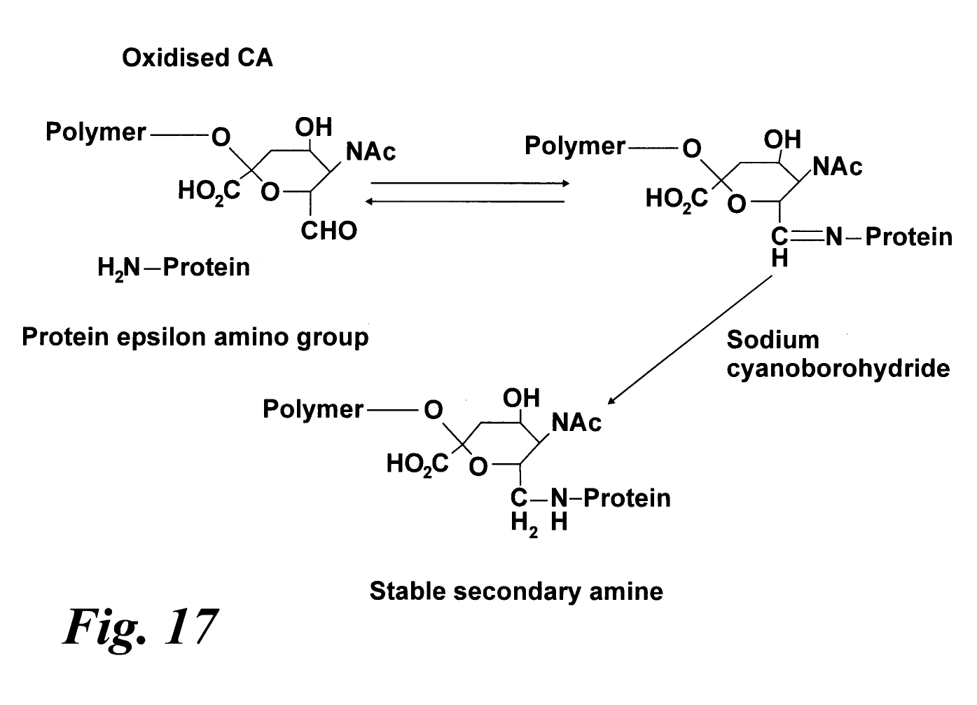

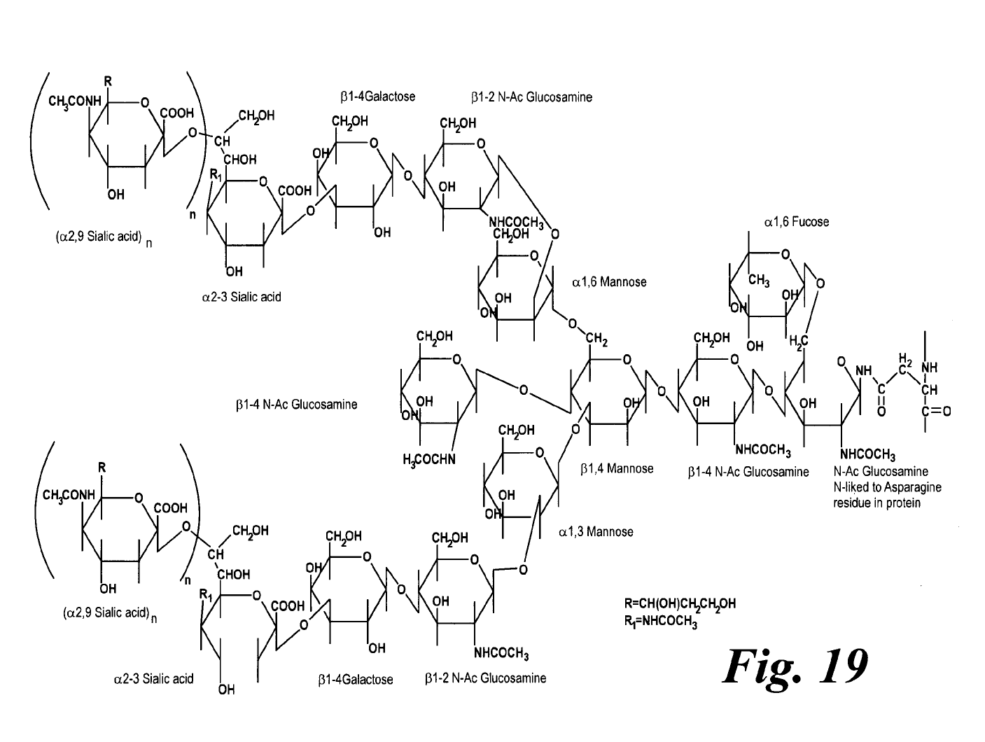

The key differences between natural/recombinant polysialylation (FIG. 19) and chemical polysialylation (FIG. 17) are:
1. The sugar polymer in natural/recombinant polysialylation is attached to asparagine residues rather than lysine or in some cases cysteine residues for chemical polysialylation.
2. The PSA molecule is only added to the protein after the naturally-occurring core glycosylation (N-Ac-Glucosamine/Mannose/Galactose) is added whereas chemical methods just attach PSA polymer without using the core glycosylation.
3. The linkage for natural/recombinant polysialylation is an amide/peptide bond rather than a secondary amine bond.
4. Natural/recombinant polysialylation requires naturally occurring glycosidic bonds, whereas the chemical method involves removing carbons 8 and 9 from the terminal end of the PSA polymer, thereby oxidizing it to an aldehyde which then reacts with the protein amine group catalysed by sodium borohydride. This essentially places the PSA chain in the reverse orientation to recombinant/naturally occurring PSA.

Preferably the first protein or domain thereof is associated with the second protein or domain thereof containing a natural polysialylation site by either conjugation or fusion.

The first protein or domain thereof is typically an active protein having a desired function, properties or structure.

Advantageously the molecule provided in step (i) is provided by expression of the molecule in a host cell. Preferably step (ii) occurs in the host cell by the cell containing a polysialyltransferase enzyme.

One embodiment of the invention is that an unmodified first protein or domain thereof is modified to include a domain comprising a natural polysialylation site.

Preferably the second protein or domain thereof containing a natural polysialylation site and the first protein or domain thereof contains at least one glycosylation motif (Asn-X-Thr/Ser)

Preferably the first protein or domain thereof is an antibody, ligand or enzyme. Conveniently the first protein is an antibody and advantageously the first protein is an scFv.

The invention can apply to the use of any protein which is naturally polysialylated (Table 2) including human proteins and modified forms thereof and non human homologues.

TABLE 2
Naturally polysialylated proteins which could
be used to make therapeutic fusion proteins

| Naturally polysialylated proteins | Refs |
|---|---|
| Neural cell adhesion molecule (NCAM) | 34 |
| Alpha-subunit of voltage-gated sodium channel | 35 |
| CD36 scavenger receptor | 36 |
| ST8SSia IV/PST polysialyltransferase (PST) | 37, 87 |
| ST8Sia II/STX polysialyltransferase (STX) | 88, 89 |
| Capsid of E. coli strain K1 | 20, 90 |
| Capsid of Neisseria meningitidis group B | 20, 91 |
| Fish egg glycoprotein | 92 |

In particular the invention can be performed using Neural Cell adhesion molecule (NCAM) and modified forms thereof.

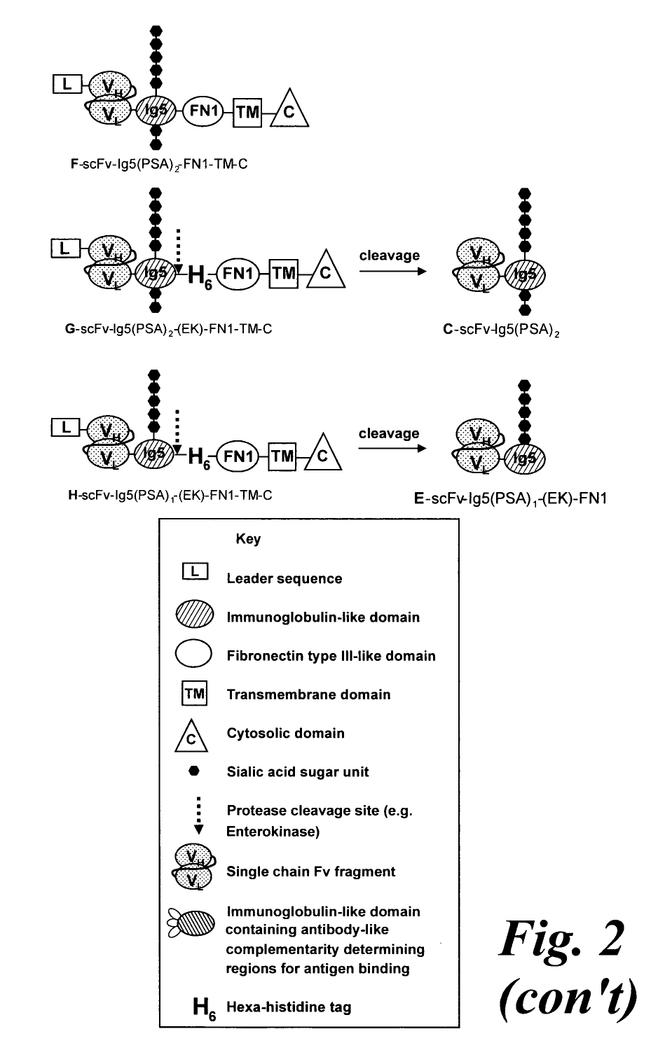

NCAM is an adhesion molecule that mediates adhesion through homophilic and heterophilic interactions leading to the activation of signalling pathways [38]. NCAM is a multi-domain receptor of the immunoglobulin superfamily consisting of 5 immunoglobulin (Ig)-like domains, 2 fibronectin type-III (FN_{III}) like domains, a trans-membrane domain and a cytosolic domain. NCAM is glycosylated throughout, but it is polysialylated only on the Ig5 domain at two [39] possibly three [40] sites (FIG. 2).

Removal of the PSA on NCAM weakens NCAM-NCAM interactions and also eliminates NCAM-independent cell interactions. These changes lead to neurite outgrowth, impaired axon guidance/pathfinding and cell migration. NCAM both enhances intermembrane repulsion and abolishes NCAM-mediated and clatherin-mediated membrane interactions [41].

Polysialic acid is highly expressed in embryos and neonate, but down-regulated in the adult, with expression confined to specialized areas in the brain where neurogenesis and cell migration are needed [35]. Experiments involving PST, STX or NCAM deficient mice have shown that the PSA on NCAM plays an important role in maintaining plasticity in particular areas of the adult central nervous system required for certain behaviour, learning and memory functions [42].

Previous research has shown that NCAM does not have to be membrane-bound to be polysialylated [44]. It was also demonstrated that polysialylation was a protein-specific event with the minimal domains needed for polysialylation being the Ig5 and FN_{III}-1 domains [44]. Further research provided evidence that the FN_{III}-1 domain is recognised by host cell polysialyl-transferases which enzymatically attaches PSA chains onto the Ig-5 domain. A more detailed study showed that other fibronectin-like domains cannot substitute for the FN_{III}-1 domain and that a critical acid patch on the surface of the FN_{III}-1 domain was the likely recognition area [39].

Conveniently the polysialylated domain(s) of the polysialylated molecule is the fifth immunoglobulin domain (Ig5) of NCAM.

Preferably the polysialylated molecule further comprises the first type-III fibronectin-like domain (FN_{III}-1) of NCAM.

In one embodiment of the invention the polysialylated molecule comprises a plurality of Ig5 domains and in an alternative embodiment the polysialylated molecule comprises a plurality of Ig5 and a plurality of FN_{III}-1 domains.

Advantageously the conjugated active protein or modified polysialylated protein exhibits altered polysialylation levels, size and/or mass; immunogenicity, blood half-life, proteolytic stability, chemical or thermal stability, tissue specificity, binding properties, catalytic activity, neutralization functions and agonistic or antagonistic receptor binding functions in comparison to the unconjugated active protein or unmodified naturally polysialylated protein and wherein the altered function may be an increase or a decrease.

Optionally, the polysialylated molecule also comprises one or more additional sequences selected from the list of: secretion signal sequences; membrane anchoring sequences (e.g. transmembrane domains or GPI-anchors); protease cleavage sites, domains for aiding detection and/or purification (e.g. hexahistidine sequence).

Advantageously the process includes the step of cleaving the expressed fusion protein to remove at least one non-polysialylated domain.

In a second aspect of the invention there is provided a polysialylated molecule that is obtained from or obtainable by the method of the first aspect of the invention.

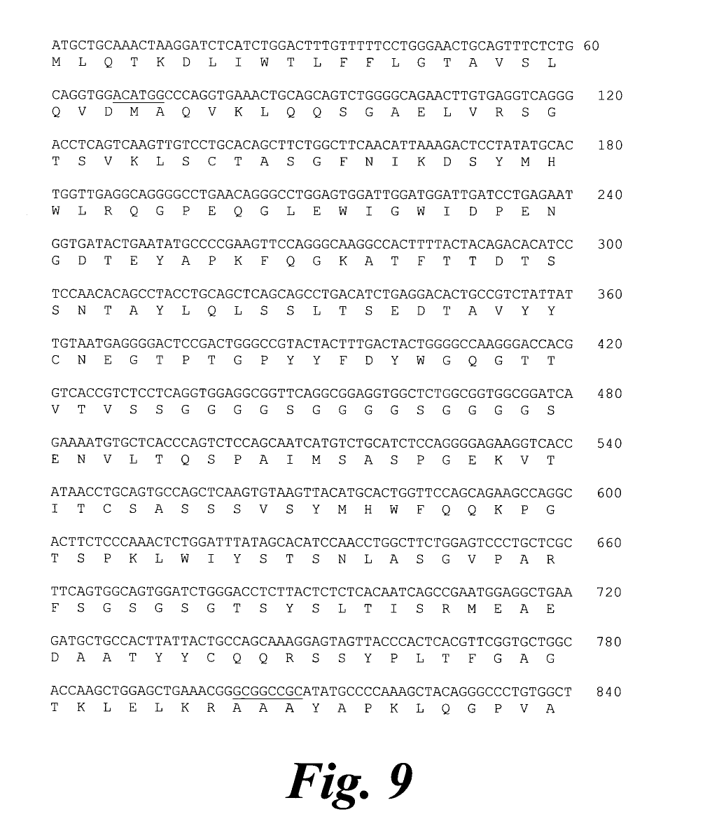

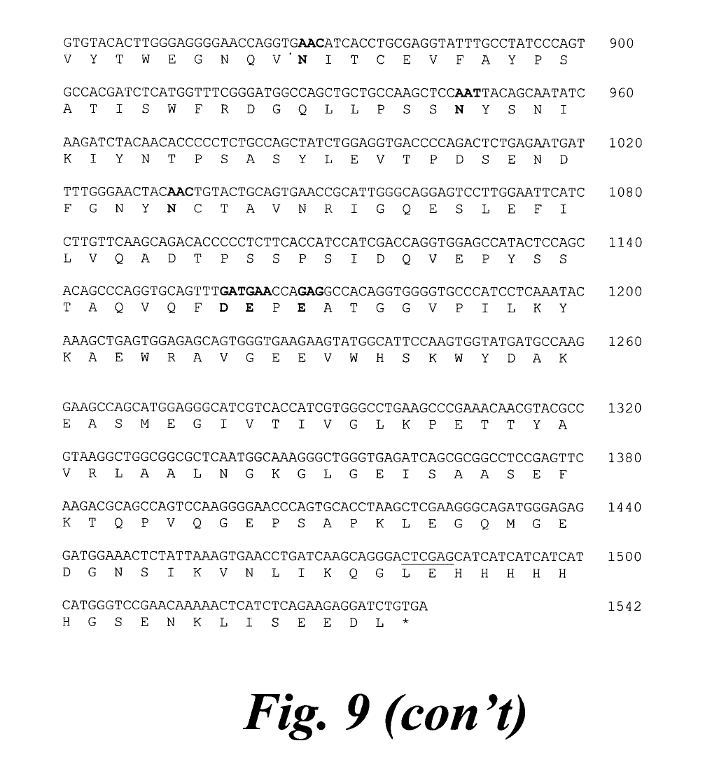

Preferably, the polysialylated molecule has the amino acid sequence of FIG. 9.

In a third aspect of the invention there is provided a nucleic acid having a nucleotide sequence encoding the polysialylated molecule of the second aspect of the invention.

Preferably the nucleic acid has the nucleotide sequence of FIG. 9.

In a fourth aspect of the invention there is provided an expression vector containing a nucleotide sequence encoding the polysialylated molecule of the second aspect of the invention.

Preferably the expression vector comprises the nucleotide sequence encoding the polysialylated molecule is that of FIG. 9.

In a fifth aspect of the invention there is provided a host cell producing a polysialylated molecule as defined in the second aspect of the invention, resulting from expression of the nucleotide sequence encoding the polysialylated molecule.

Preferably the nucleotide sequence expressed by the host cell is that of FIG. 9

In a sixth aspect of the invention there is provided a composition comprising the polysialylated molecule as defined in the second aspect of the invention and a pharmaceutically acceptable carrier, excipient and/or diluent.

In a seventh aspect of the invention there is provided a polysialylated molecule as defined in the second aspect of the invention or a composition as defined in the sixth aspect of the invention for use in the treatment of disease.

In a eighth aspect of the invention there is provided a use of a polysialylated molecule as defined in the second aspect of the invention in the manufacture of a medicament for the treatment and/or diagnosis and/or prevention of solid cancer (e.g. breast, prostate, lung, renal, colorectal), disseminated cancers (e.g. lymphomas and leukaemias), infectious diseases (e.g. malaria, leishmanaisis, meningitis, botulinum poisoning, E. coli, influenza, HIV, hepatitis), narcotics poisoning (e.g. cocaine) and cardiovascular diseases (blood clots, heart disease).

In an ninth aspect of the invention there is provided the use of a polysialylated molecule as defined in the second aspect of the invention in a screening assay.

Preferably, the screening assay comprises identifying antibodies, antibody fragments or antibody derivatives that are able to bind a target molecule.

### Meanings of Terms Used

By “a naturally polysialylated domain associated with an first protein or domain thereof” we include conjugates and fusion proteins. The polysialiylated and acive portions of the molecule may be adjacent or one may be incorporated within the other (for example see FIG. 10 in which the CDR domain is incorporated into the polysialylated domain).

By “naturally polysialylated” we mean that the domain that is polysialylated comprises a sugar chain N-linked onto an asparagine residue of the domain. The PSA chain is added onto a core carbohydrate sequence so it differs completely from any chemically made protein-PSA conjugates Natural polysialylation does not include chemical polysialylation or recombinant polysialylation.

By “chemical polysialylation” we mean the chemical modification of the reducing or non-reducing end of a PSA chain (usually from bacterial sources) to form reactive aldehyde or maleimide groups. This then reacts with amines (N-terminal residue, Lysine, Arginine) or thiols (cysteine) respectively to form a covalent bond (see FIG. 17 adapted from WO2005/016974).

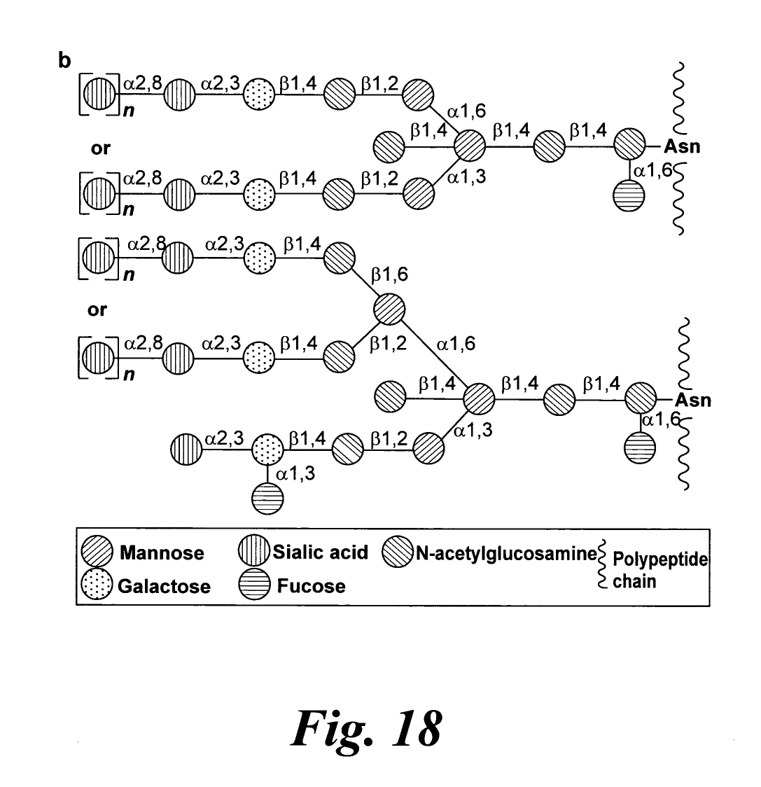

By “recombinant polysialylation” we mean the addition of di- and tri-antennary core N-glycans (2/3 branches) to form a different amide bond with the nitrogen of the asparagines. The PSA is then added onto the galactose residues of this core. So the overall structure of the sugar is very different from a naturally polysialylated molecule (see FIG. 18).

The terms “nucleotide sequence” or “nucleic acid” or “polynucleotide” or “oligonucleotide” are used interchangeably and refer to a heteropolymer of nucleotides or the sequence of these nucleotides. These phrases also refer to DNA or RNA of genomic or synthetic origin which may be single-stranded or double-stranded and may represent the sense or the antisense strand, to peptide nucleic acid (PNA) or to any DNA-like or RNA-like material. In the sequences herein A is adenine, C is cytosine, T is thymine, G is guanine and N is A, C, G or T (U). It is contemplated that where the polynucleotide is RNA, the T (thymine) in the sequences provided herein is substituted with U (uracil). Generally, nucleic acid segments provided by this invention may be assembled from fragments of the genome and short oligonucleotide linkers, or from a series of oligonucleotides, or from individual nucleotides, to provide a synthetic nucleic acid which is capable of being expressed in a recombinant transcriptional unit comprising regulatory elements derived from a microbial or viral operon, or a eukaryotic gene.

The terms “polypeptide” or “peptide” or “amino acid sequence” refer to an oligopeptide, peptide, polypeptide or protein sequence or fragment thereof and to naturally occurring or synthetic molecules. A polypeptide “fragment,” “portion,” or “segment” is a stretch of amino acid residues of at least about 5 amino acids, preferably at least about 7 amino acids, more preferably at least about 9 amino acids and most preferably at least about 17 or more amino acids. To be active, any polypeptide must have sufficient length to display biological and/or immunological activity.

The term “domain” as used herein denotes a polypeptide chain or part thereof that can fold independently into a stable tertiary structure and has a specific function. For example, an antibody binding site consisting of CDR sequences forms a stable tertiary structure with the function of binding to a target antigen. Therefore a domain is any structurally or functionally distinct part of a larger molecule.

The terms “purified” or “substantially purified” as used herein denotes that the indicated nucleic acid or polypeptide is present in the substantial absence of other biological macromolecules, e.g., polynucleotides, proteins, and the like. In one embodiment, the polynucleotide or polypeptide is purified such that it constitutes at least 95% by weight, more preferably at least 99% by weight, of the indicated biological macromolecules present (but water, buffers, and other small molecules, especially molecules having a molecular weight of less than 1000 daltons, can be present).

The term “isolated” as used herein refers to a nucleic acid or polypeptide separated from at least one other component (e.g., nucleic acid or polypeptide) present with the nucleic acid or polypeptide in its natural source. In one embodiment, the nucleic acid or polypeptide is found in the presence of (if anything) only a solvent, buffer, ion, or other component normally present in a solution of the same. The terms “isolated” and “purified” do not encompass nucleic acids or polypeptides present in their natural source.

The term “recombinant,” when used herein to refer to a polypeptide or protein, means that a polypeptide or protein is derived from recombinant (e.g., microbial, insect, or mammalian) expression systems. “Microbial” refers to recombinant polypeptides or proteins made in bacterial or fungal (e.g., yeast) expression systems. As a product, “recombinant microbial” defines a polypeptide or protein essentially free of native endogenous substances and unaccompanied by associated native glycosylation. Polypeptides or proteins expressed in most bacterial cultures, e.g., E. coli, will be free of glycosylation modifications; polypeptides or proteins expressed in yeast will have a glycosylation pattern in general different from those expressed in mammalian cells.

The term “expression vector” refers to a plasmid or phage or virus or vector, for expressing a polypeptide from a DNA (RNA) sequence. An expression vehicle can comprise a transcriptional unit comprising an assembly of (1) a genetic element or elements having a regulatory role in gene expression, for example, promoters and often enhancers, (2) a structural or coding sequence which is transcribed into mRNA and translated into protein, and (3) appropriate transcription and translation initiation and termination sequences. Structural units intended for use in yeast or eukaryotic expression systems preferably include a leader sequence enabling extracellular secretion of translated protein by a host cell. Alternatively, where recombinant protein is expressed without a leader or transport sequence, it may include an amino terminal methionine residue. This residue may or may not be subsequently cleaved from the expressed recombinant protein to provide a final product.

The term active protein shall be taken to refer to a protein having a particular effector function that is therapeutically, diagnostically, chemically or biotechnologically desirable. Examples of active proteins include but are not limited to antibodies, enzymes and receptors.

The term “antibody” shall be taken to refer to any one of an antibody, an antibody fragment, or antibody derivative. It is intended to embrace wildtype antibodies (i.e. a molecule comprising four polypeptide chains), synthetic antibodies, recombinant antibodies or antibody hybrids, such as, but not limited to, a single-chain modified antibody molecule produced by phage-display of immunoglobulin light and/or heavy chain variable and/or constant regions, or other immunointeractive protein capable of binding to an antigen in an immunoassay format that is known to those skilled in the art.

The term “antibody derivative” refers to any modified antibody molecule that is capable of binding to an antigen in an immunoassay format that is known to those skilled in the art, such as a fragment of an antibody (e.g. Fab or Fv fragment), or a modified antibody molecule that is modified by the addition of one or more amino acids or other molecules to facilitate coupling the antibodies to another peptide or polypeptide, to a large carrier protein or to a solid support (e.g. the amino acids tyrosine, lysine, glutamic acid, aspartic acid, cysteine and derivatives thereof, NH₂-acetyl groups or COOH-terminal amido groups, amongst others).

The term “ScFv molecule” refers to any molecules wherein the V_{H} and V_{L} partner domains' are linked via a flexible oligopeptide.

The terms “selective binding” and “binding selectivity” indicates that the variable regions of the antibodies of the invention recognise and bind polypeptides of the invention exclusively (i.e., able to distinguish the polypeptide of the invention from other similar polypeptides despite sequence identity, homology, or similarity found in the family of polypeptides), but may also interact with other proteins (for example, S. aureus protein A or other antibodies in ELISA techniques) through interactions with sequences outside the variable region of the antibodies, and in particular, in the constant region of the molecule. Screening assays to determine binding selectivity of an antibody of the invention are well known and routinely practiced in the art. For a comprehensive discussion of such assays, see Harlow et al. (Eds), Antibodies A Laboratory Manual; Cold Spring Harbor Laboratory; Cold Spring Harbor, N.Y. (1988), Chapter 6. Antibodies that recognise and bind fragments of the polypeptides of the invention are also contemplated, provided that the antibodies are first and foremost selective for, as defined above, full-length polypeptides of the invention. As with antibodies that are selective for full length polypeptides of the invention, antibodies of the invention that recognise fragments are those which can distinguish polypeptides from the same family of polypeptides despite inherent sequence identity, homology, or similarity found in the family of proteins.

The term “binding affinity” includes the meaning of the strength of binding between an antibody molecule and an antigen.

### PREFERRED EMBODIMENTS

Examples embodying certain preferred aspects of the invention will now be described with reference to the following figures in which:—

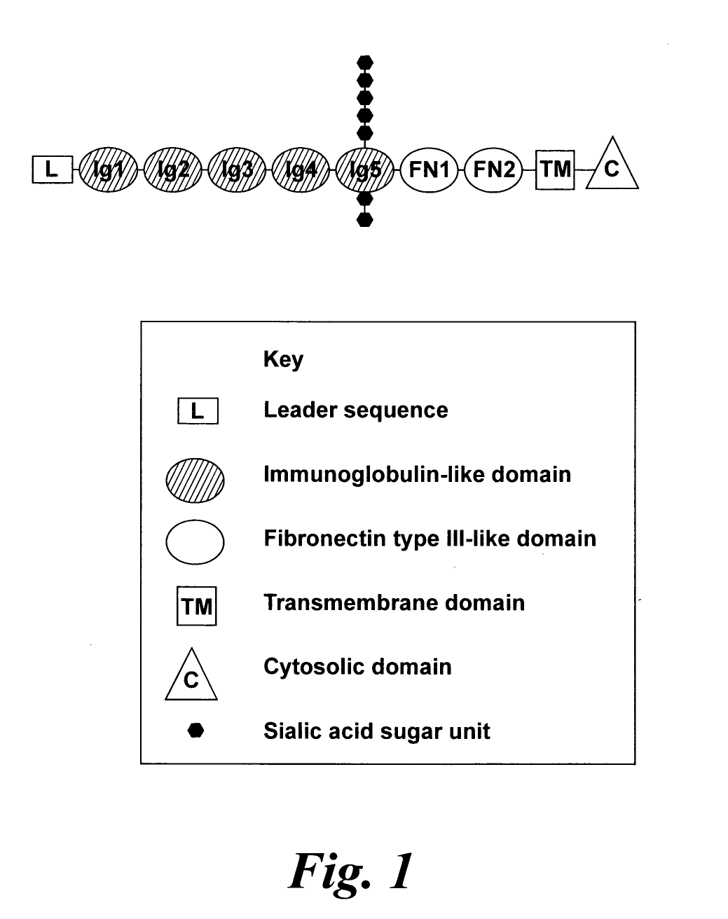

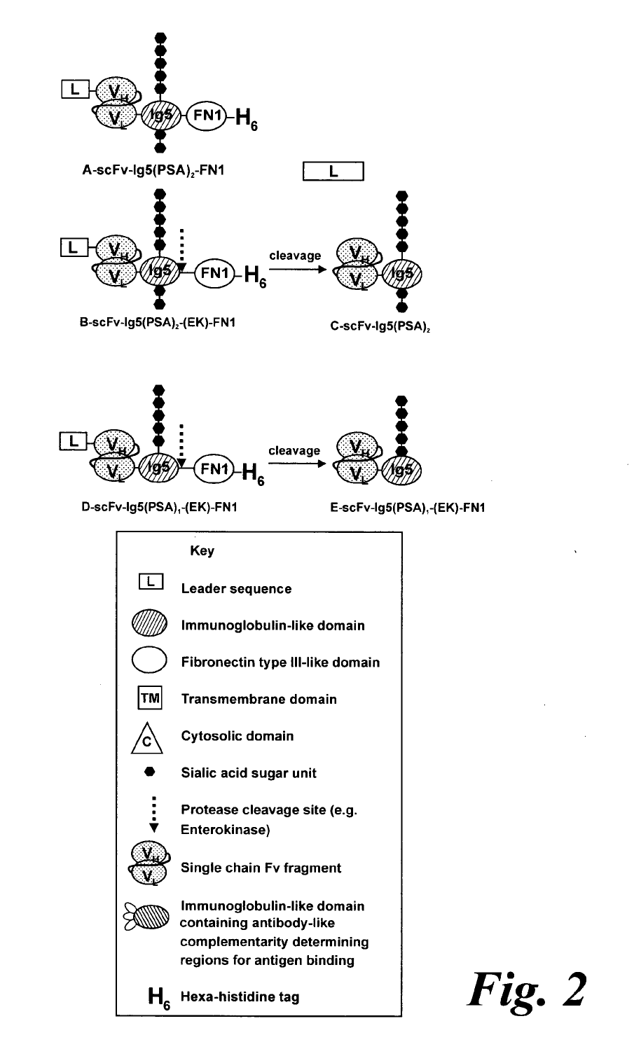

FIG. 1—Wildtype NCAM

Schematic diagram of wild-type human, full length NCAM, showing position of polysialylation.

FIG. 2—Polysialylated ScFv-NCAM fusion

Schematic diagram of proposed single-chain Fv-NCAM fusion proteins. These all contain Ig5 and FN-1 domains which are solubly expressed, expressed as transmembrane proteins with the potential to be cleaved to yield the scFv-Ig5 domains alone.

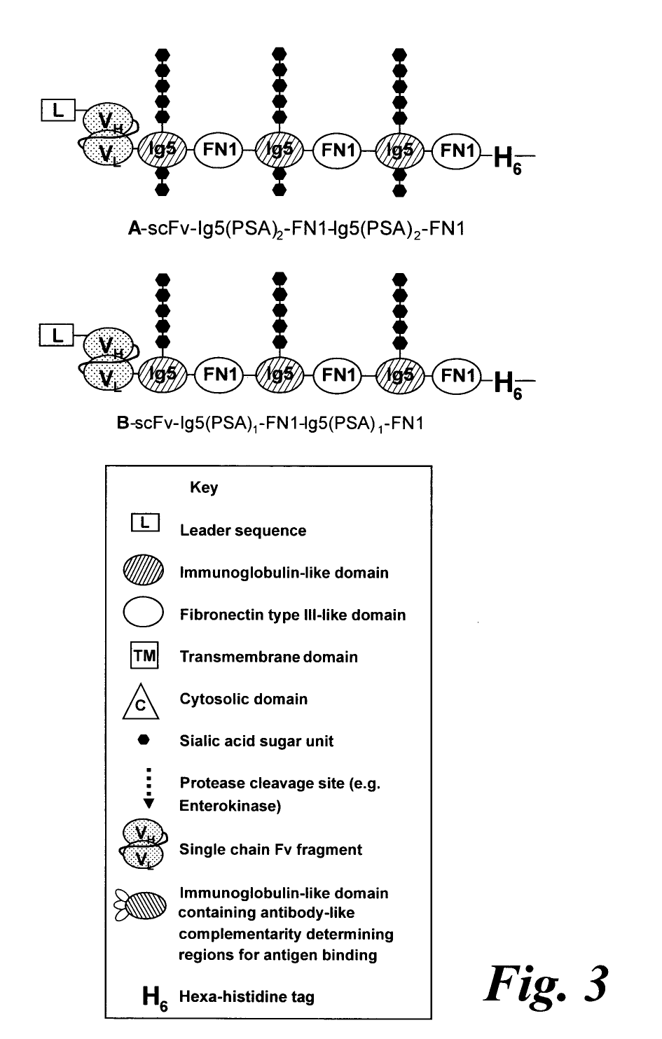

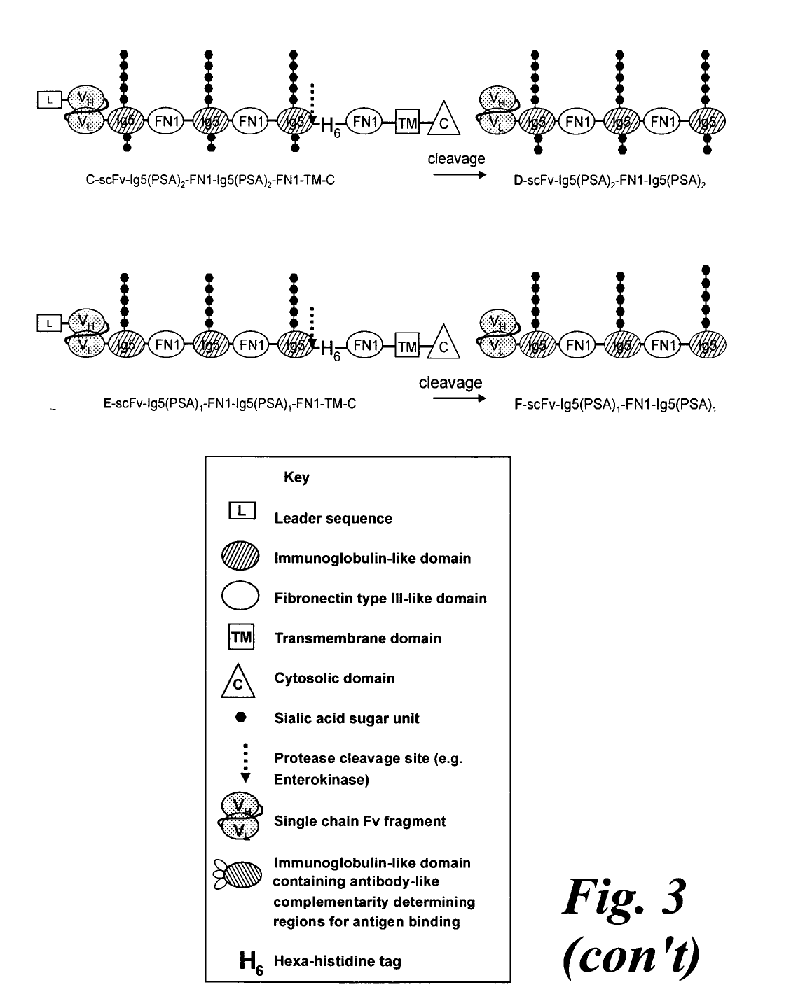

FIG. 3—Polysialylated ScFv-NCAM fusion with multiple polysialylation and nonsilaylation domains of NCAM

Schematic diagram of scFv-NCAM fusion proteins with multiple Ig5-FN-1 polysialylation domains. These all contain Ig5 and FN-1 domains which are solubly expressed, expressed as transmembrane proteins with the potential to be cleaved to yield the scFv-Ig5 domains alone.

FIG. 4—Polysialylated ScFv-NCAM fusion with multiple polysialylation domains

Schematic diagram of scFv-NCAM fusion proteins with multiple Ig5 polysialylation domains. The Ig5 domain is repeated to add further polysialic acid onto the fusion protein. These all contain Ig5 and FN-1 domains which are solubly expressed, expressed as transmembrane proteins with the potential to be cleaved to yield the scFv-Ig5 domains alone.

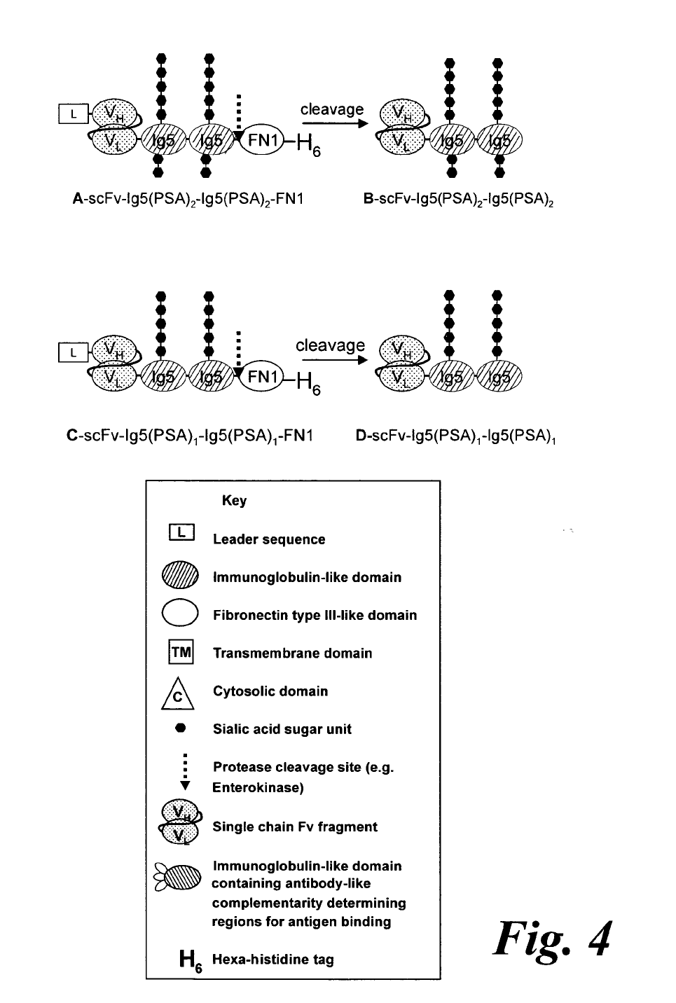

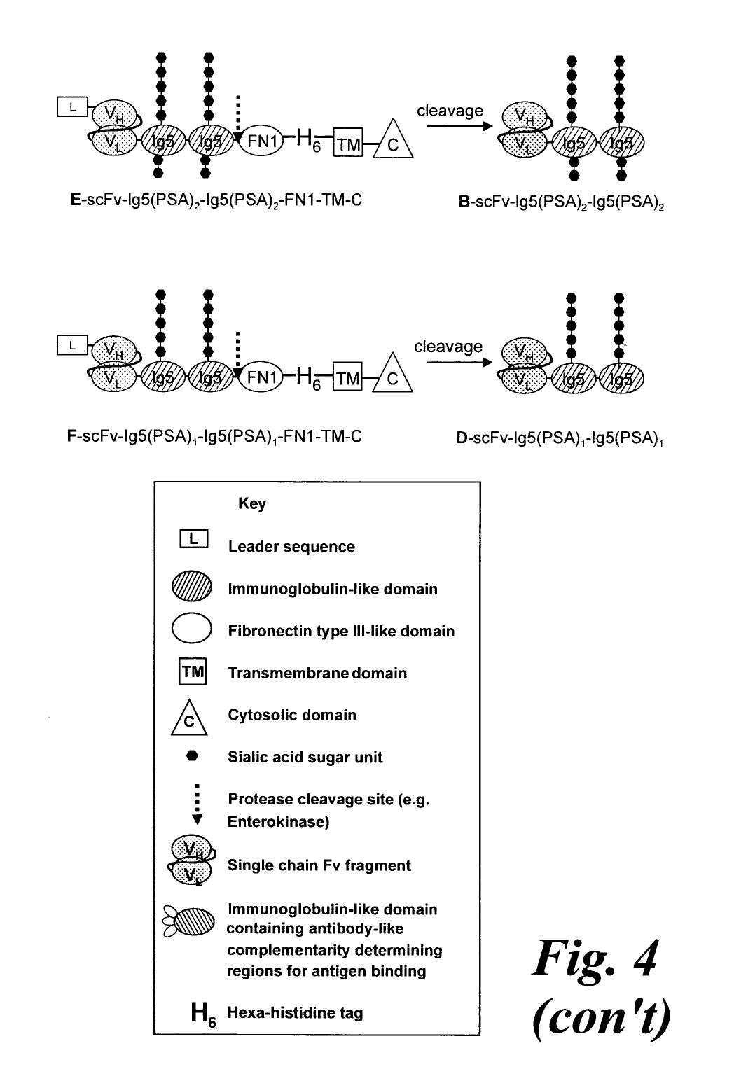

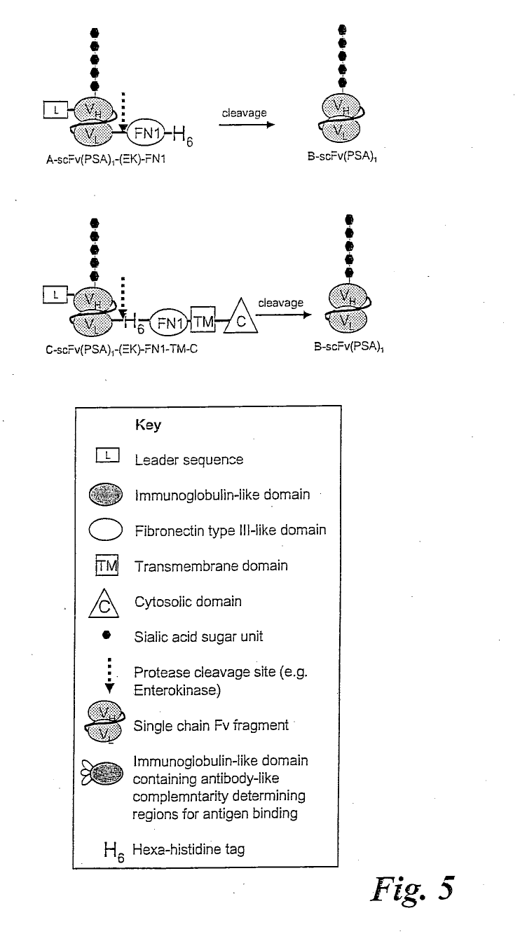

FIG. 5—Polysialylated ScFv derived from NCAM fusion

Schematic diagram of proposed single-chain Fv-NCAM fusion proteins. The scFv can be engineered to accept the PSA chains as if it were an NCAM Ig5 domain. These all contain FN-1 domains which are solubly expressed, expressed as transmembrane proteins with the potential to be cleaved to yield the scFv domains alone.

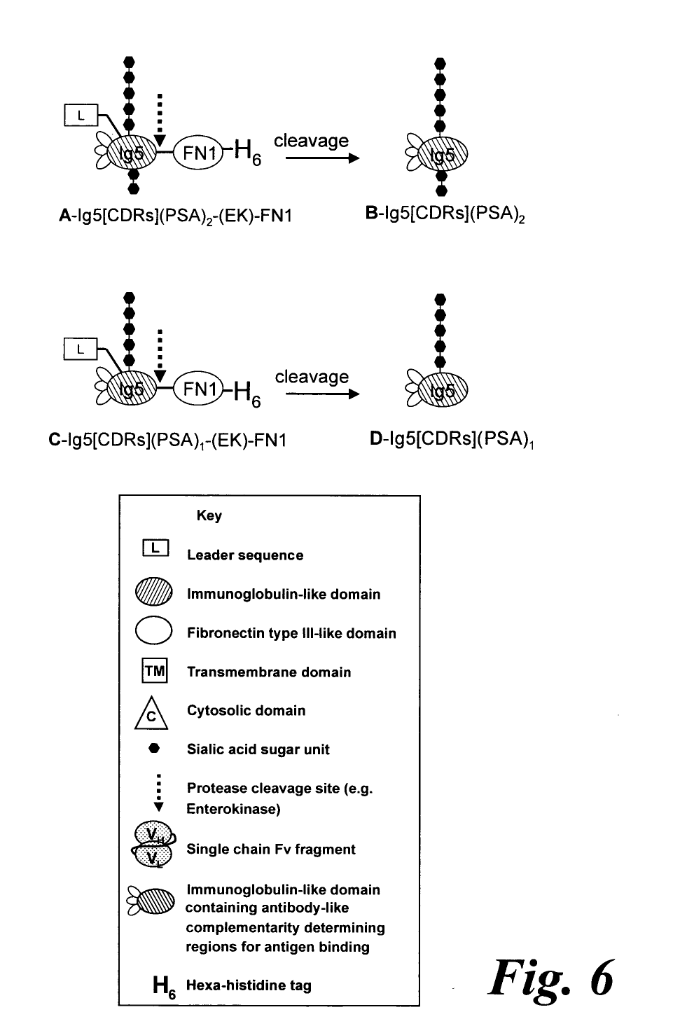

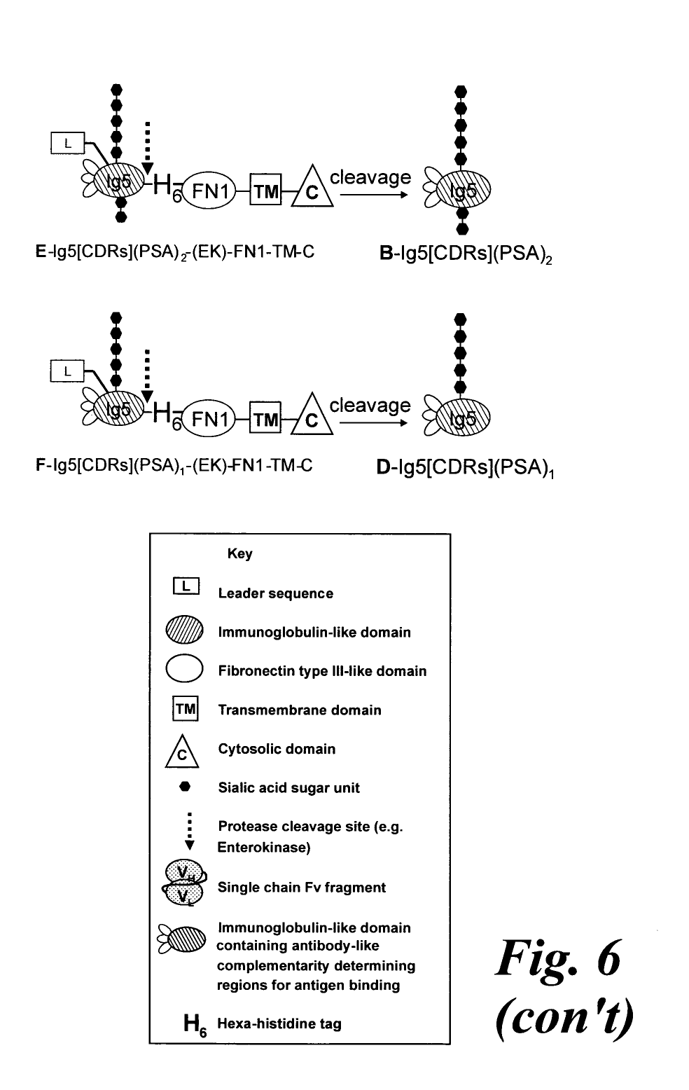

FIG. 6—Polysialylated modified NCAM domains

Schematic diagram of proposed antigen-binding-NCAM fusion proteins. The Ig5 domain can be engineered to possess binding properties like conventional antibodies. These all contain Ig5 and FN-1 domains which are solubly expressed, expressed as transmembrane proteins with the potential to be cleaved to yield the Ig5 domain alone.

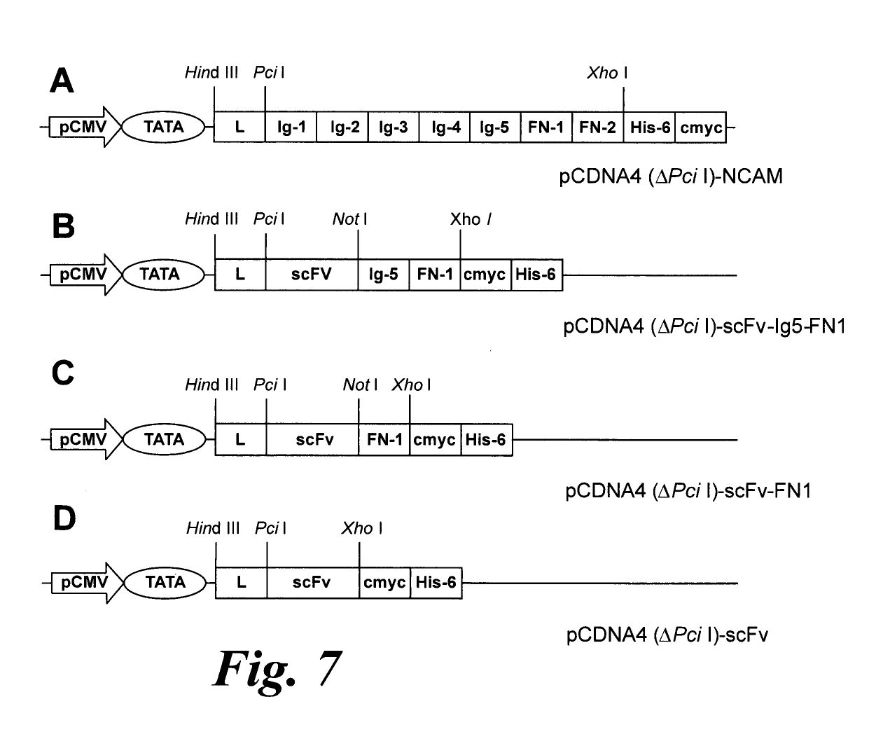

FIG. 7—Schematic diagram of 4 NCAM-fusion protein as DNA constructs

(A) Full length, soluble NCAM with his and myc tags.

(B) scFv-Ig5-FN_{III}-1 with his and myc tags.

(C) scFv-FN_{III}-1 with his and myc tags.

(D) scFv only with his and myc tags.

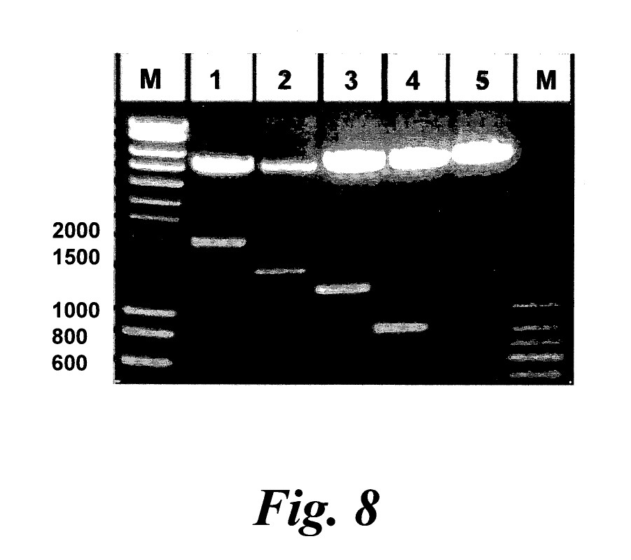

FIG. 8—Restriction digest (Hind III/Xho I) analyses of NCAM fusion clones

(M) markers

(1) pcDNA4(ΔPci I)-NCAM, 5.3 kb (vector)+2 kb (gene)

(2) pcDNA4(ΔPci I)-scFv-Ig5-FN1, 5.3 kb (vector)+1.5 kb (gene)

(3) pcDNA4(ΔPci I)-scFv-FN1, 5.3 kb (vector)+1.2 kb (gene)

(4) pcDNA4(ΔPci I)-scFv, 5.3 kb (vector)+0.9 kb (gene)

(5) pcDNA4(ΔPci I), 5.3 kb (vector)

FIG. 9—Annotated DNA Sequence of NCAM Fusion Gene in pcDNA4(ΔPci I)-scFv-Ig5-FN1

1-69=human NCAM secretion sequence, ending with a hybrid Pci I/Nco I site (underlined)

70-801=Anti-CEA scFv ending with Not I site (underlined)

801-810=linker

811-1092=human Ig5 domain with N-linked polysialylation sites in bold

1093-1479=Human FN1 domain ending with Xho I site (underlined) with acid recognition motif residues in bold

1480-1485=linker

1486-1503=hexahistidine tag

1504-1509=linker

1510-1542=myc tag/stop codon

FIG. 10—SDS-PAGE of NCAM fusion proteins expressed in Dulbecco's Modified Eagle Medium (DMEM)

(M) Protein markers

(1) scFv-Ig5-FN1, calculated protein MW=53442 Da, observed MW=72500 Da

(2) scFv-FN1, calculated protein MW=43856 Da, observed MW=50000 Da

(3) scFv, calculated protein MW=33040 Da, observed MW=3400 Da

FIG. 11—SDS-PAGE of NCAM fusion proteins expressed in CHO media

(M) Protein markers

(1) scFv-Ig5-FN1, calculated protein MW=53442 Da, observed MW=72500 Da

(2) scFv-FN1, calculated protein MW=43856 Da, observed MW=50000 Da

(3) scFv, calculated protein MW=33040 Da, observed MW=3400 Da

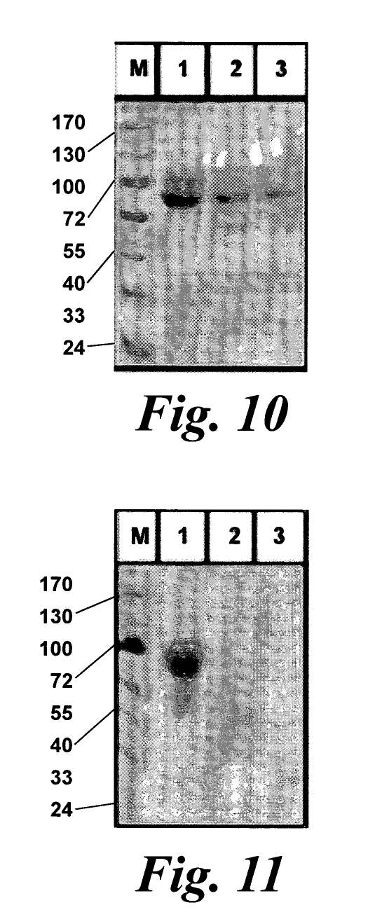

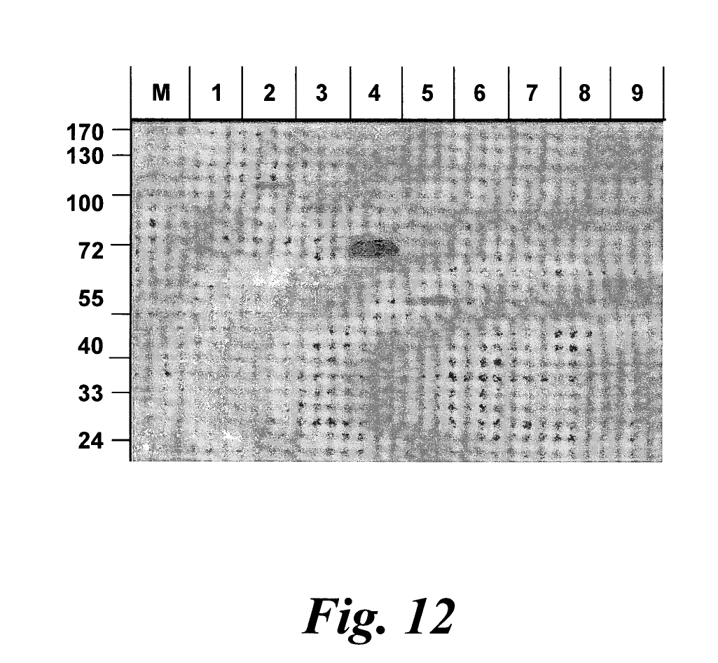

FIG. 12—Western Blot analyses of transiently expressed NCAM fusion clones

(M) markers

(1) pcDNA4(ΔPci I)

(2) pIg-NCAM-Fc

(3) pcDNA4(ΔPci I)-NCAM-Fc

(4) pcDNA4(ΔPci I)-scFv-Ig5-FN1

(5) pcDNA4(ΔPci I)-scFv-FN1

(6) pcDNA4(ΔPci I)-scFv

(7) No DNA control

(8) No lipofectin control

(9) media only

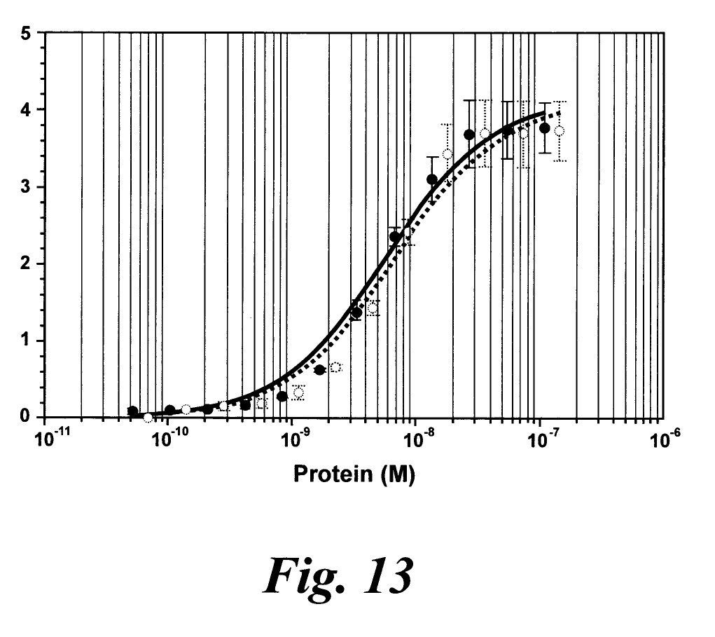

FIG. 13—ELISA of anti-carcinoembryonic antigen (anti-CEA) scFv and anti-CEA scFv-Ig5-FN1

ELISA of an anti-CEA scFv (black) and the scFv-Ig5-FN1 fusion protein (grey) on immobilised CEA. The approximate Kds are 6×10⁻⁹ M for the scFv and 7×10⁻⁹ M for the fusion suggesting that the additional domains incorporated do not significantly affect the binding affinity.

FIG. 14—Neuramidase treatment

(M) Protein markers

(1) scFv-Ig5-FN1−neuramidase

(2) scFv-Ig5-FN1+neuramidase

(3) scFv-FN1−neuramidase

(4) scFv-FN1+neuramidase

(5) scFv−neuramidase

(6) scFv+neuramidase

(7) Transferrin−neuramidase

(8) Transferrin+neuramidase

(9) Media only

Arrow A shows the shift in molecular weight after neuramidase treatment for the scFV-Ig5-FN1 protein which is not seen in the scFv-Fn1 or scFv proteins (B and C)

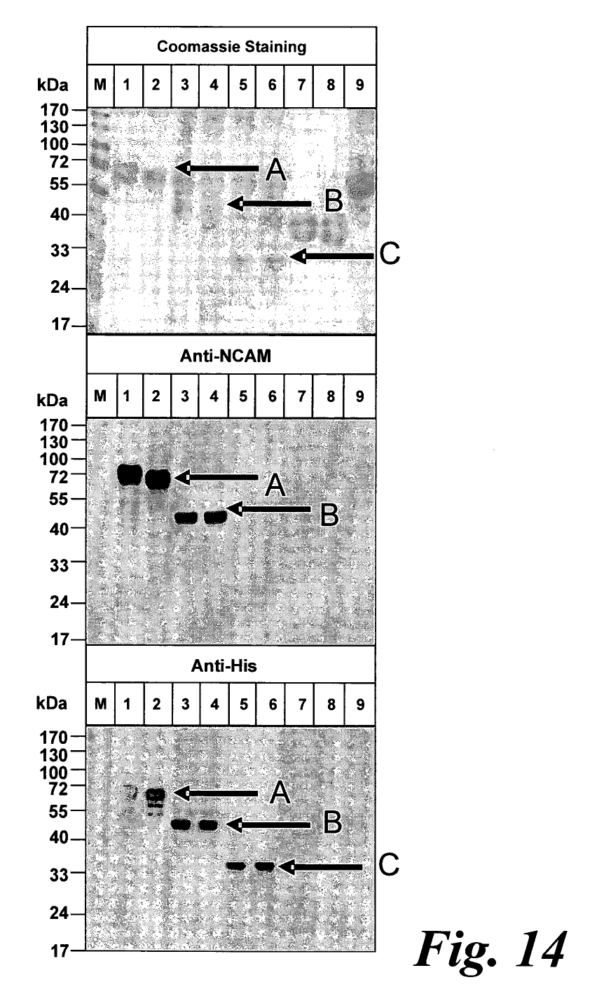

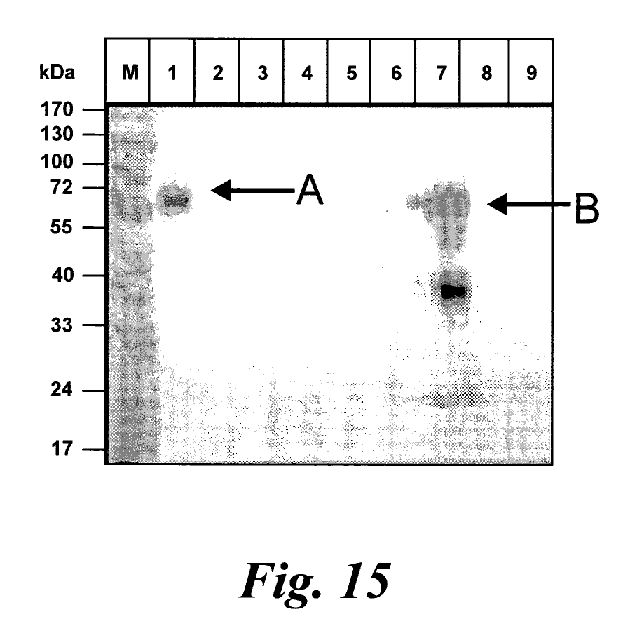

FIG. 15—Anti-sialic acid analysis of scFv fusion constructs before and after neuraminidase treatment.

Equimolar amounts of CHO derived purified proteins; scFv-Ig5-Fn 1 (lanes 1 & 2), scFv-Fn 1 (lanes 3 & 4) and scFv (lanes 5 & 6) were loaded. Positive sialylated control protein transferrin was also analysed (lanes 7 & 8), whilst CHO media alone was used as a negative control (lane 9). These were treated with (even number lanes) or without (odd number lanes) neuraminidase at 37° C. overnight. SDS-PAGE followed by anti-sialic Western blot analysis revealed that only the scFv-Ig5-Fn1 and transferrin control were significantly sialylated prior to treatment, while no detection in post-treatment samples indicated the cleavage of sialic acid by neuraminidase. No sialic acid was found to be associated with either of the two other constructs; scFv-Fn 1 or scFv, prior to neuraminidase activity and the CHO media negative control indicated no background activity.

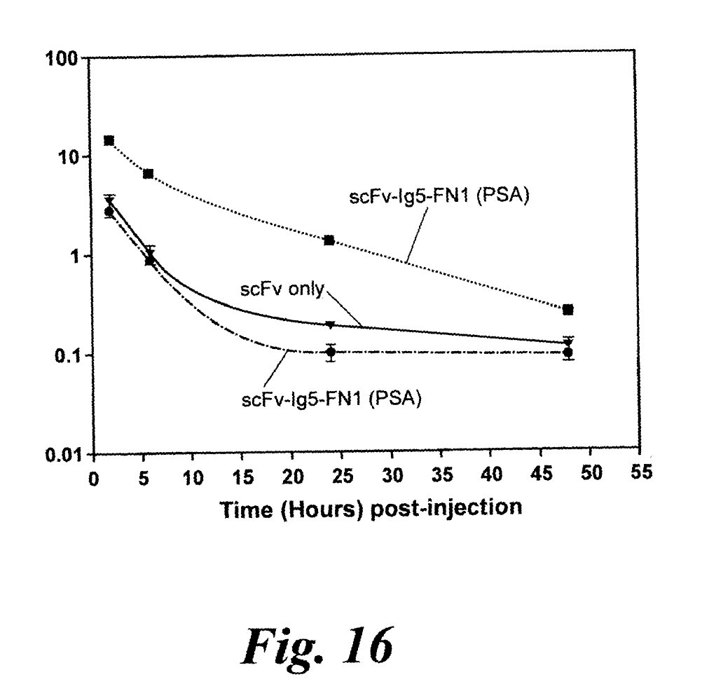

FIG. 16—Blood Clearance Pharmacokinetics of scFv, scFv-Ig5-FN1 (polysialylated, +PSA and desialylated, −PSA) in nude mice.

Ten micrograms of each pure protein was radiolabelled using the Iodogen method and injected IV into the tail veins of 12 mice each. Mice were sacrificed at 2, 6, 24 and 48 hrs and the amount of labelled protein in the blood was determined by gamma counting. The blood clearance profile is plotted and shows that the polysialylated protein has a significantly longer half life with ‘area under the curve’ values (representing blood exposure) of 23.4, 120.1 and 17.6% hour/g for each construct respectively.

FIG. 17—Chemical polysialylation

FIG. 17 shows the structure of PSA chains when added chemically to proteins. (adapted from WO 2005/016974).

FIG. 18—Recombinant polysialylation

FIG. 18 shows the structure of PSA chain-protein conjugates when the SA is added in a recombinant system (adapted from Kleene & Schachner (2004) Nature Reviews Neuroscience 5 pp 195-208).

FIG. 19—Natural/Recombinant polysialylation (chemical structure)

Chemical structure of PSA chain as attached to a protein via N-linked glycosylation at an asparagine residue.

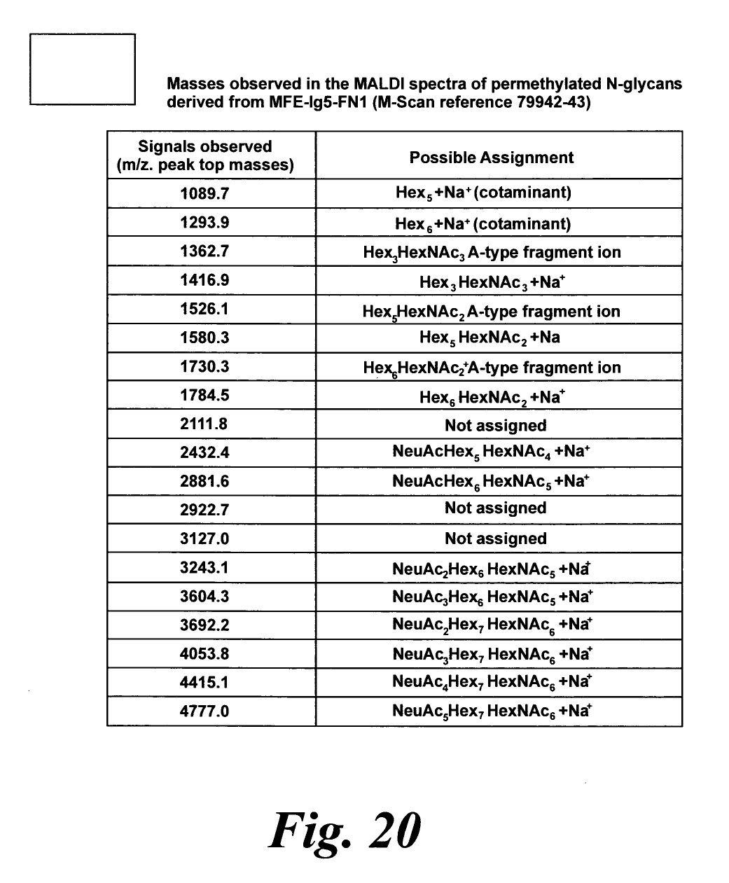

FIG. 20—Masses observed in the MALDI spectra of permethylated N-glycans derived from MFE-Ig5-FN1

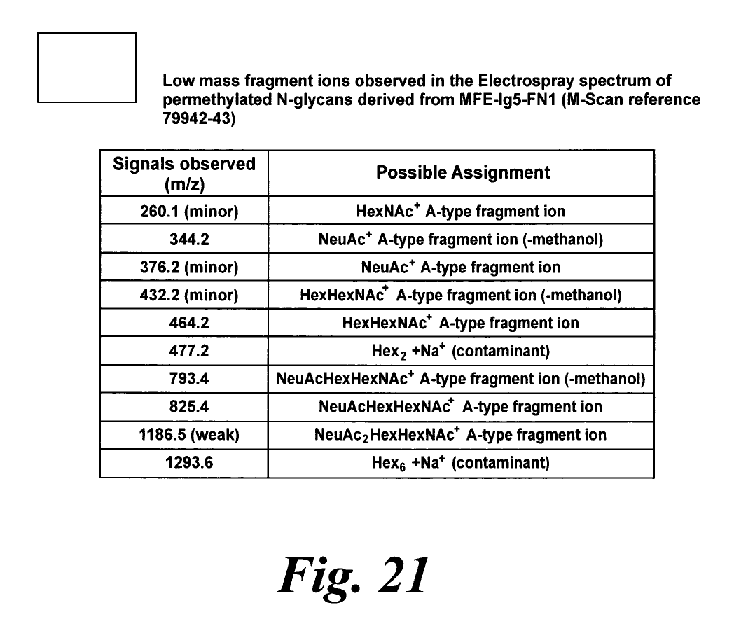

FIG. 21—Low mass fragment ions observed in the Elementary spectrum of permethylated N-glycans derived from MFE-Ig5-FN1

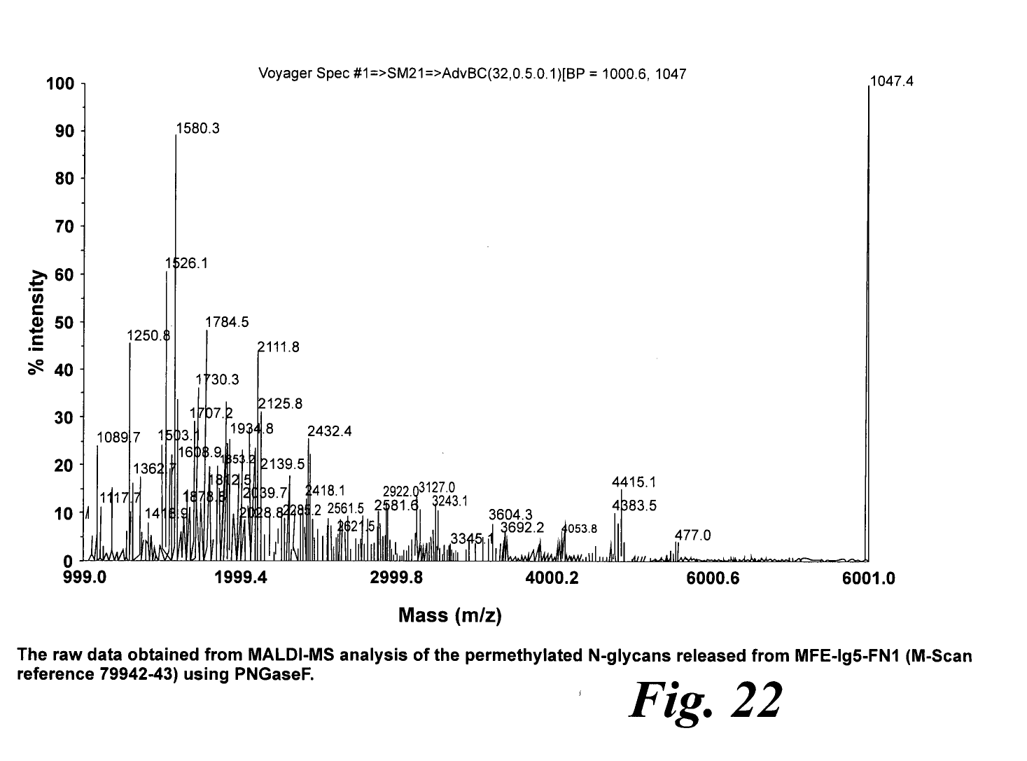

FIG. 22—Data obtained from MALDI-MS analysis of the permethylated N-glycans released from MFE-Ig5-FN1 using PNGase F

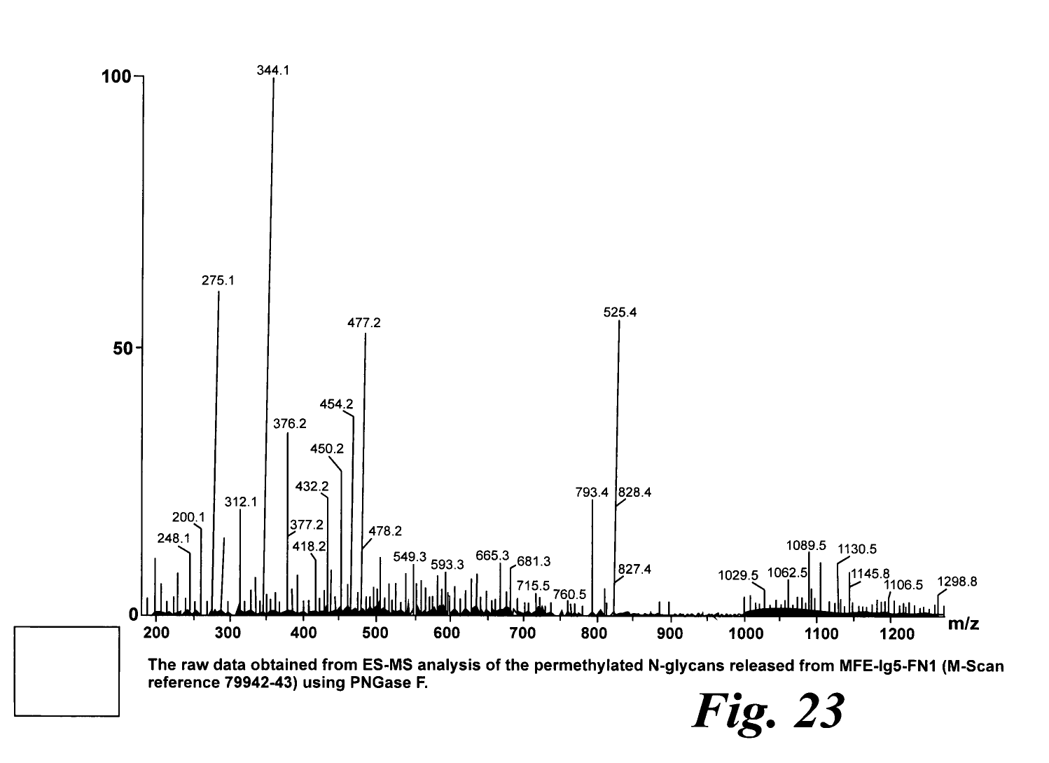

FIG. 23—Data, obtained from ES-MS analysis of the permethylated N-glycans released from MFE-Ig5-FN1 using PNGase F

### EXAMPLE 1

### Construction of scFv-Ig5-FN_{III}-1 Gene Fusion

Molecular cloning, using established molecular biology techniques [93] was used to produce 4 DNA constructs in the mammalian expression vector pcDNA4 (Invitrogen Ltd).

The Pci I site was removed from the pcDNA4 vector backbone (position 3335-3340) by silent site-directed mutagenesis (Stratagene Quikchange method [Kunkel (1985) Rapid and efficient site-specific mutagenesis without phenotypic selection. Proc Natl Acad Sci USA. 1985 January; 82(2):488-92.]) using the oligonucleotide primers 5′ GCT GGC CTT TTG CTC AGA TGG TCT TTC CTG CGT TAT CCC C 3′ and 5′ GGG GAT AAC GCA GGA AAG ACC ATG TGA GCA AAA GGC CAG C 3′.

The full length human wild-type NCAM was derived from the pIg-NCAM construct [92] which contains the gene for the soluble form of NCAM with a Immunoglobulin-kappa secretion signal.

A two-step PCR reaction was used to amplify the NCAM up to the FN1 domain, possessing a 5′ Pci I site and 3′ Xho site, using the oligonucleotide, primers 5′ GCT ACT AAG CTT GCC GCC AGC ATG GTG CAA ACT AAG GAT CTC ATC TGG 3′, 5′ GCT GAT CTC CCC CTG GCT GGG AAA CAT GTC CAC CTG CAG AGA AAC TGC AGT TCC 3′, 5′ GCC GTA GTC TCG AGT CCT GTA GAT GTC CTG AAC ACA AAA TGA GC 3′ using the mega primer method.

This PCR product was ligated into pcDNA4(DPci I) as a Hind III/Xho I fragment to make pcDNA4(DPci I)-NCAM. The Ig5-FN1 subgene were PCR amplified from pcDNA4(DPci I)-NCAM using the oligonucleotide primers 5′ CCT ATT AAC ATG TCA TCT GGA GCA GCG GCC GCA TAT GCC CCA AAG CTA CAG GGC CCT GTG G 3′ and 5′ CGT AGT CTC GAG TCC CTG CTT GAT CAG GTT CAC TTT AAT AG 3′ and replaced the Pci I/Xho I fragment of pCDNA4(DPci I)-NCAM to form pCDNA4(DPci I)-Ig5-FN1.

The scFv was inserted into this as an Nco I/Not I PCR product from a pHEN vector carrying an anti-CEA scFv. This plasmid was called pCDNA4(DPci I)-scFv-Ig5-FN1.

The FN1 subgene was PCR amplified from pcDNA4(DPci I)-NCAM using oligonucleotide primers 5′ CCT ATT AAC ATG TCA TCT GGA GCA GCG GCC GCA TTC ATC CTT GTT CAA GCA GAC ACC CCC TC 3′ and 5′ CGT AGT CTC GAG TCC CTG CTT GAT CAG GTT CAC TTT AAT AG 3′ and replaced the Pci I/Xho I fragment of pcDNA4(DPci I)-NCAM to form pcDNA4(DPci I)-FN1.

The scFv was inserted into this as an Nco I/Not I digestion product from a pHEN vector carrying an anti-CEA scFv. This plasmid was called pcDNA4(DPci I)-scFv-FN1. The scFv was digested as a Hind III/Not I fragment and ligated into the Hind III/Not I site of pcDNA4(ΔPci I) to form the plasmid pcDNA4(ΔPci I)-scFv.

Schematic diagrams of the 4 constructs are shown in FIG. 7. Each clone was verified by DNA restriction digest analyses. FIG. 8 shows each of the 4 constructs digested with Hind III/Xho I giving the expected molecular weight. The annotated DNA sequence of the NCAM fusion gene in pcDNA4(ΔPci I)-scFv-Ig5-FN1 is shown in FIG. 9.

### Example 2

### Expression & Purification of a scFv-Ig5-FN_{III}-1 Fusion Protein

The 5 clones pcDNA4(APci I)-NCAM, pcDNA4(APci I)-scFv-Ig5-FN1, pcDNA4(APci I)-scFv-FN1, pcDNA4(APci I)-scFv, pcDNA4(APci I) were transfected in NB2 murine neuroblastoma cells using Fugene (Invitrogen) according to the manufacturers conditions. 3 microlitres of the Fugene reagent was added to 97 microlitres of unsupplemented media and 1 microgram of DNA. This mixture was incubated for 15 minutes before being added to cells and left overnight. The transfectants were allowed to express protein for 48 hours.

Complete Dulbecco's Modified Eagle Medium (DMEM) was used for one set of transfections and protein free CHO media (medium specially developed for growth of Chinese Hamster Ovary cells) was used for another. The expressed proteins were purified by immobilised metal affinity chromatography (IMAC) using Talon® according to the manufacturer's instructions.

SDS-PAGE analysis of three DNA constructs from the DMEM transfectant (FIG. 10) and CHO media transfectants (FIG. 11) shows the presence of the fusion proteins. There are contaminating serum proteins in the DMEM media purified samples, whereas the CHO media purified samples are pure.

The predicted molecular weights have been determined from the amino acid sequence (FIGS. 10 & 11) and the observed molecular weights are shown (FIGS. 10 & 11). There is close agreement, except an almost 20,000 Da (20 kDa) difference for the scFv-Ig5-FN1 protein. The observed difference are expected to be due to the different levels of glycosylation and further polysialylation.

Western Blotting of the 5 transiently-expressing constructs with an anti-NCAM (FN1-domain specific) antibody confirms the presence of the fusion proteins as predicted (FIG. 12).

### Example 3

### ELISA of an scFv-Ig5-FN_{III}-1 Fusion Protein

Carcinoembryonic antigen (CEA) was coated onto a 96-well microtitre plate (2 μg/ml) in PBS overnight and used in an ELISA. Serial dilutions of anti-CEA scFv and anti-CEA scFv-Ig5-FN1 proteins were added. Binding was allowed to proceed for 1 hr at room temperature and detection was by murine anti-His, rabbit-anti mouse Ig-HRPO followed by development by BM blue substrate.

The binding signal of both clones are visualised and plotted in FIG. 13. The binding profile was fitted to a sigmoidal curve using SigmaPlot®. As can be seen, there is no significant difference in the binding affinity of either clone. The affinities were estimated as 6 nM for the anti-CEA scFv and 6.8 nM for the scFv-Ig5-FN1 fusion protein.

### Example 4

### Neuramidase Treatment of a scFv-Ig5-FN_{III}-1 Fusion Protein

Three NCAM fusion proteins were expressed in NB2 cells in DMEM media and the semi-pure protein (after Talon® purification) (Porath, J. (1992) Protein Express. Purif. 3:263-281.) was treated with neuramidase enzyme (0.2 units, overnight at 37 degrees).

The samples are analysed before and after treatment by SDS-PAGE (FIG. 14A), Anti-NCAM Western Blot (FIG. 14B) and Anti-His Blot (FIG. 14C). The scFv-Ig5-FN1 fusion protein can be seen to decrease in molecular weight as seen by a shift in migration, after neuramidase treatment. This suggests that this protein is highly sialylated and most likely polysialylated due to the 2-3 glycosylation sequences present.

There is no visible shift in molecular weight for the scFv-FN1 (detectable anti-NCAM and anti-His) or scFv (detectable anti-His only). The molecular weight shift was estimated to be 5000 Da, which if present on two sites of sialylation, corresponds to some 15-18 residues i.e. PSA chains of at least 8 sialic acid units.

### Example 5

### Direct Polysialylation Detection in a scFv-Ig5-FN_{III}-1 Fusion Protein

One microgram of pure scFv-Ig5-FN1, scFv-FN1 and scFv expressed from the pcDNA4 vectors in protein-free CHO media was analysed by SDS-PAGE followed by Western Blotting with anti-sialic acid antibodies, before and after treatment with 0.2 units of neuramidase (overnight at 37° C.). The anti-sialic acid antibodies detect the sialic acid component from NCAM or similar glycoproteins where the number of sialic acid units are greater than 10. It can be seen that only the scFv-Ig5-FN1 was sialylated. The negative controls (scFv-FN1 and scFv) do not exhibit sialylation. A positive control protein (transferrin) is also seen to be sialylated (FIG. 15).

### Example 6

### In Vivo Pharmacokinetics of a scFv-Ig5-FN_{III}-1 Fusion Protein

One hundred micrograms of pure scFv-Ig5-FN1 was desialylated with neuramidase (1 unit, overnight at 37° C.). This protein was repurified on Talon® resin to remove contaminants. This desialylated protein (DS-scFv-Ig5-FN1) was radiolabelled with 125I using the Iodgen method, along with 100 μg of sialylated scFv-Ig5-FN1 and 100 μg of scFv. Five micrograms of each radiolabelled protein was injected, IV into the tail veins of 12 BALB/C nude mice. Groups of three mice, from each sample, were sacrificed at 2, 6, 24 and 48 hours. The amount of radiolabelled protein remaining in the blood was determined by radio-active gamma counting and compared to the initial dose injected.

These values are expressed as % injected dose/gram blood over time. It can be seen that the scFv-Ig5-FN1 containing PSA has a longer blood half-life compared to the same protein without PSA (after neuramidase treatment) or the free scFv alone (FIG. 16).

The relative areas under the curve, representing blood exposure were 17.6 (DS-scFv-Ig5-FN1), 23.4 (scFv) and 120.1 (scFv-Ig5-FN1), representing an increase in the presence of the scFv in the blood of seven-fold due to the presence of the PSA chain.

### Example 7

### scFv-Ig5-FN_{III}-1 Fusion Protein Comprising Multiple Ig5-FN_{III}-1 Repeats

An alternative fusion protein to the scFv-Ig5-FN_{III}-1 of Example 1 can be constructed using multiple Ig5-FN_{III}-1 domains linked together to give increased size and polysialylation (FIG. 3).

These fusion proteins can be made using the methods of Examples 1 and 2, differing only by constructing vectors that contain multiple repeats of the nucleotide sequence encoding the Ig5-FN_{III-}1 domains.

This can be achieved by molecular cloning of a PCR product containing the Ig5-FN1 domains. Using PCR primers ‘TTTGGGCTCGAGTATGCCCCAAAGCTA’ and ‘TTTGGGCTCGAGTCCCTGCTTGATCAG’ a cassette encoding the Ig5-FN1 domains flanked by Xho I sites is produced, which can be digested with Xho I and ligated into the Xho I site in the pcDNA4(ΔPci I)-scFv-Ig5-FN1 vector. Clones with the Ig5-FN1 in the correct orientation are determined by DNA sequencing. Further domains can be inserted to produce more Ig5-FN1 containing fusion proteins by repeating the above step.

These fusion proteins, can also have mutant Ig5 domains with altered levels of polysialylation (FIGS. 3B, 3E & 3F) engineered by the addition or removal of glycosylation motifs (e.g. Asn-X-Thr/Ser), either be expressed solubly (FIGS. 3A & 3B) or be membrane tethered (FIGS. 3C & 3E), and may contain proteolytic cleavage sites to allow the removal of the FN_{III}-1 domain (FIGS. 3C & 3E leading to FIGS. 3D & 3F).

### Example 8

### scFv-Ig5-FN_{III}-1 Fusion Protein Comprising Multiple Ig5 Repeats

A further alternative fusion protein to the scFv-Ig5-FN_{III}-1 of Example 1 can be constructed using multiple Ig5 domains linked to give increased size and polysialylation but without the presence of multiple and in some cases any FN_{III}-1 domains (FIG. 4).

This can be achieved by molecular cloning of a PCR product containing the Ig5 domain. Using PCR primers ‘TTTGGGACTGATTATGCCCCAAAGCTA’ and ‘TTTGGGACTGATTGCTTGAACAAGGATGAA’ a cassette encoding the Ig5 domain flanked by Cla I sites is produced, which can be digested with Cla I and ligated into the Cla I site (which has been introduced by site-directed mutagenesis using the primers ‘ATCCTTGTTACTGATGACACCCC’ and ‘GGGGGTGTCATCAGTAACAAGGAT’) in the pcDNA4(ΔPci I)-scFv-Ig5-FN1 vector. Clones with the Ig5-FN1 in the correct orientation are determined by DNA sequencing. Further domains can be inserted to produce more Ig5-FN1 containing fusion proteins by repeating the above step.

These fusion proteins, can also have mutant Ig5 domains with altered levels of polysialylation (FIGS. 4C, 4D & 4F) engineered by the addition or removal of glycosylation motifs (e.g. Asn-X-Thr/Ser), either be expressed solubly (FIGS. 4A & 4C) or be membrane tethered (FIGS. 4E & 4F), and may contain proteolytic cleavage sites to allow the removal of the FN_{III}-1 domain (FIGS. 4E & 4F leading to FIGS. 4B & 4D).

### Example 9

### Polysialylated scFv Derived from ScFv-FN_{III}-1 Fusion Protein

A further alternative polysialylated protein to the scFv-Ig5-FN_{III}-1 of Example 1 can be constructed using an antibody fragment such as a scFv linked directly to the FN_{III}-1 domain (FIG. 5). The scFv fragment should be modified to possess glycosylation motifs (e.g. Asn-X-Thr/Ser) in similar or appropriate topological places to that found in the NCAM-Ig 5 domain.

One such position is approximately 42 residues from a key acid motif in the scFv-FN_{III}-1 within the scFv sequence. In this example oligonucleotide primers can be used to introduce a glycosylation motif into the scFv at this position into the vector pcDNA4(ΔPci I)-scFv-FN1. The primers used can be ‘TATTACTGCCAGAACTGTACTAGTTACCCACTC’ and ‘GAGTGGGTAACTAGTACAGTTCTGGCAGTAATA’. This construct is expressed and characterised as described above.

In this example, the scFv then becomes the substrate for the polysialyltransferase enzymes and accepts the PSA chains instead of the Ig5 domain.

These proteins, like above either be expressed solubly (FIG. 5A) or be membrane tethered (FIG. 5C), and may contain proteolytic cleavage sites to allow the removal of the FN_{III}-1 domain (FIG. 5C leading to FIG. 5B).

### Example 10

### Polysialylated Modified NCAM Domains

A further alternative polysialylated protein to the scFv-Ig5-FN_{III}-1 of Example 1 can be constructed using a modified Ig5 domain that has a desired activity. One possible embodiment of this example is an Ig5 domain that has been modified either by rational site-directed mutagenesis [85] or random mutagenesis, followed by a selection process if appropriate [86, 87] to form an Ig5 domain capable of binding antigen (FIG. 6). Strategies to obtain antigen binding Ig5 domain include homology modelling between antigen-binding human V-domains and the human NCAM Ig5 domain to identify which residues could be mutated in order to bind to an antigen, or phage display of the whole Ig5 domain to select for binders after error-prone PCR mutagenesis.

This modification could be the result of the introduction of antigen-binding loops similar to the complementarity determining regions (CDRs) found in antibodies. In other words the modified Ig5 domain has been modified to include an antigen binding domain via the inclusion of CDR sequences which form a tertiary structure with a specified function of binding antigen.

In addition to the modification of the Ig5 domains to include an “active site”, the Ig5 domains can be further mutated to have altered levels of polysialylation (FIGS. 6C & 6F) by the addition or removal of glycosylation motifs (e.g. Asn-X-Thr/Ser). These proteins, like above, can either be expressed solubly (FIGS. 6A & 6C) or be membrane tethered (FIGS. 6E & 6F) with protease cleavage sites to remove unwanted domains (FIGS. 6E & 6F leading to FIGS. 6B & 6D).

### Example 11

### Further Modifications of Polysialylated Compounds and their Synthesis

For all of the polysialylated proteins described above, the growth and expression conditions can be manipulated to alter the yields of polysialylated fusion protein and the level of polysialylation on each recombinant protein. This can include the use of chemicals or drugs to alter glycosylation pathways, expression time, addition of exogenous PSA or sialic acid, addition of heterologous genes to modulate the sialic acid biosynthetic pathway, etc.

One example is the use of the drug Valproic acid. This has been shown to increase the level of expression of the ST8SiaIV polysialyltransferase enzyme, resulting in increased levels of NCAM polysialylation [Beecken, W-D et al. (2005). Int Immunopharm. 5, 757-769]. Another example is the heterologous expression of the enzyme UDP-N-acetylglucosamine 2-epimerase/N-acetyl-mannosamine-kinase (GNE), a key enzyme in the biosynthesis of sialic acid. The expression of a feedback mutant form of this enzyme or a sialic acid precursor such as N-acetyl mannosamine can lead to increased levels of sialic acid and polysialylation [Bork, K et al (2005) Febs Letts 579, 5079-83.

### Example 12

### Confirmation of Polysialylation of the scFv-Ig5-FN1 Fusion Protein by Mass Spectrometry

### Methods

Analyses were carried out using procedures involving the determination of retention time and mass as a diagnostic for structure. Analyses were performed using a PerSeptive Biosystems Voyager STR DE-MALDI-TOF mass spectrometer. The procedures and analyses were carried out by M-SCAN Ltd, 3 Millars Business Centre, Fishponds close, Wokingham, UK.

### Sample Preparation

Two hundred micrograms of pure scFv-Ig5-FN1 was prepared as described in example-2 and concentrated to 0.2 mg/ml in phosphate buffered saline.

### Reduction/Carboxymethylation

Reduction/carboxymethylation was performed on the sample using dithiothreitol (DTT) 4-fold molar excess over the number of disulphide bridges (30 mins at 37° C.) followed by iodoacetic acid (IAA-5-fold molar excess over the amount of DTT for 30 mins at room temperature) in tris-acetate buffer at pH 8.5. The products of the reduction/carboxymethylation reaction were purified using Millipore's Microcon spin cartridges and eluted with 100 μL of 50 mM ammonium bicarbonate pH 8.4.

### Tryptic Digestion

Digestion was performed for 5 h at 37° C. using TPCK treated trypsin (1:50 enzyme to substrate ratio) in 50 mM ammonium bicarbonate pH 8.4. The digest was lyophilised.

### Peptide N-Glycosidase F Digestion

The tryptically cleaved peptide/glycopeptide mixture was treated with 4 units of the enzyme peptide N-glycosidase F in 50 mM ammonium bicarbonate pH 8.4 for 16 h at 37° C. The resulting products were purified using a C18 Sep-Pak, N-glycans were eluted using 5% aq. acetic acid. The N-glycan fraction was lyophilised, permethylated using the sodium hydroxide (NaOH)/methyl iodide (MeI) procedure and analysed by Delayed Extraction-Matrix Assisted Laser Desorption Ionisation-Time of Flight-Mass Spectrometry (DE-MALDI-TOF MS) and Electrospray Mass Spectrometry (ES-MS).

### Delayed Extraction-Matrix Assisted Laser Desorption Ionisation-Time of Flight-Mass Spectrometry (DE-MALDI-TOF-MS)

MALDI-TOF mass spectrometry was performed using a Voyager-DE STR Biospectrometry Research Station laser-desorption mass spectrometer using Delayed Extraction (DE) technology. Dried permethylated glycans were redissolved in methanol:water (80:20) and analysed using a matrix of 2,5-dihydroxybenzoic acid. Angiotensin and ACTH fragments were used as external calibrants.

### Electrospray Mass Spectrometry (ES-MS)

Electrospray-MS was performed using a quadrupole-orthogonal acceleration time of flight (Q-TOF) instrument using Argon as collision gas. Glu-Fibrinopeptide fragment ions in MS/MS mode were used to calibrate the instrument. Dried permethylated glycans were redissolved in methanol:0.1% TFA (80:20) before analysis.

### RESULTS AND DISCUSSION

### N-Linked Oligosaccharide Population Screening by MALDI-MS

The samples were reduced and carboxymethylated. A small amount of precipitation was observed in the reaction products which may have affected the amount of material analysed. The supernatant was removed and purified using a Microcon spin cartridge. Trypsin digestion was then performed. The lyophilised products were digested using PNGase F and then purified using a C18 Sep-Pak. The 5% aq. acetic acid (N-linked oligosaccharide containing) fraction was permethylated and DE-MALDI-TOF mass spectra were obtained using a portion of the derivatised oligosaccharides in a high mass range for molecular ions. The raw data obtained are shown in FIG. 22. Signals consistent with some under and/or over methylation were observed. This was most noticeable in the high mass complex structures. FIG. 20 lists the predominant molecular ions present in the MALDI spectra.

### N-Linked Oligosaccharide Population Screening by ES-MS

Following MALDI-MS analysis, a fraction of the permethylated N-glycans were analysed by Electrospray-Mass Spectrometry (ES-MS). The raw data is shown in FIG. 23. FIG. 21 lists the predominant fragment ions present in the Electrospray spectrum. The data obtained show fragment ions which are consistent with antennal structures expected to be present in the complex glycans detected by MALDI-MS.

### CONCLUSION

The data shows the presence of high mannose and complex N-glycan structures on the glycoprotein. The major structures present are high mannose representing early structures in N-glycanbiosynthesis. Complex structures were detected with masses consistent with bi-, tri- and tetra-antennary structures with varying levels of sialylation. Evidence of polysialylated structures has been found and these were detected at minor levels on the tetra-antennary glycans. The m/z peak at 4777 is consistent with a polysialylated multi-antennae structure. Its low, levels may be due to the incomplete processing and the low concentration of the sample used for this experiment.

### Example 13

### Pharmaceutical Formulations and Administration

A further aspect of the invention provides a pharmaceutical formulation comprising a compound according to the first aspect of the invention in admixture with a pharmaceutically or veterinarily acceptable adjuvant, diluent or carrier.

Preferably, the formulation is a unit dosage containing a daily dose or unit, daily sub-dose or an appropriate fraction thereof, of the active ingredient.

The compounds of the invention will normally be administered orally or by any parenteral route, in the form of a pharmaceutical formulation comprising the active ingredient, optionally in the form of a non-toxic organic, or inorganic, acid, or base, addition salt, in a pharmaceutically acceptable dosage form. Depending upon the disorder and patient to be treated, as well as the route of administration, the compositions may be administered at varying doses.

In human therapy, the compounds of the invention can be administered alone but will generally be administered in admixture with a suitable pharmaceutical excipient diluent or carrier selected with regard to the intended route of administration and standard pharmaceutical practice.

For example, the compounds of the invention can be administered orally, buccally or sublingually in the form of tablets, capsules, ovules, elixirs, solutions or suspensions, which may contain flavouring or colouring agents, for immediate-, delayed- or controlled-release applications. The compounds of invention may also be administered via intracavernosal injection.

Such tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compounds of the invention may be combined with various sweetening or flavouring agents, colouring matter or dyes, with emulsifying and/or suspending agents and with diluents such as water, ethanol, propylene glycol and glycerin, and combinations thereof.

The compounds of the invention can also be administered parenterally, for example, intravenously, intra-arterially, intraperitoneally, intrathecally, intraventricularly, intrasternally, intracranially, intra-muscularly or subcutaneously, or they may be administered by infusion techniques. They are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

For oral and parenteral administration to human patients, the daily dosage level of the compounds of the invention will usually be from 1 mg/kg to 30 mg/kg. Thus, for example, the tablets or capsules of the compound of the invention may contain a dose of active compound for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention.

The compounds of the invention can also be administered intranasally or by inhalation and are conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoro-ethane, a hydrofluoroalkane such as 1,1,1,2-tetrafluoroethane (HFA 134A3 or 1,1,1,2,3,3,3-heptafluoropropane (HFA 227EA3), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a compound of the invention and a suitable powder base such as lactose or starch.

Aerosol or dry powder formulations are preferably arranged so that each metered dose or “puff” delivers an appropriate dose of a compound of the invention for delivery to the patient. It will be appreciated that he overall daily dose with an aerosol will vary from patient to patient, and may be administered in a single dose or, more usually, in divided doses throughout the day.

Alternatively, the compounds of the invention can be administered in the form of a suppository or pessary, or they may be applied topically in the form of a lotion, solution, cream, ointment or dusting powder. The compounds of the invention may also be transdermally administered, for example, by the use of a skin patch. They may also be administered by the ocular route, particularly for treating diseases of the eye.

For ophthalmic use, the compounds of the invention can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the compounds of the invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

Formulations suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouth-washes comprising the active ingredient in a suitable liquid carrier.

Generally, in humans, oral or topical administration of the compounds of the invention is the preferred route, being the most convenient. In circumstances where the recipient suffers from a swallowing disorder or from impairment of drug absorption after oral administration, the drug may be administered parenterally, e.g. sublingually or buccally.

For veterinary use, a compound of the invention is administered as a suitably acceptable formulation in accordance with normal veterinary practice and the veterinary surgeon will determine the dosing regimen and route of administration which will be most appropriate for a particular animal.

### REFERENCES

[1] Walsh G (2005) Trends Biotechnol. 23, 553-8. Biopharmaceuticals: recent approvals and likely directions.
[2] Lobo E D, Hansen R J & Balthasar J P (2004). J Pharm Sci. 93, 2645-68. Antibody pharmacokinetics and pharmacodynamics.
[3] DeFelippis M R, Chance R E & Frank B H (2001). Crit. Rev Ther Drug Carrier Syst. 18, 201-64. Insulin self-association and the relationship to pharmacokinetics and pharmacodynamics.
[4] Harris J M & Chess R B (2003). Nat Rev Drug Discov. 2, 214-21. Effect of pegylation on pharmaceuticals.
[5] Batra S K, Jain M, Wittel U A, Chauhan S C & Colcher D (2002) Curr Opin Biotechnol. 13, 603-8 Pharmacokinetics and biodistribution of genetically engineered antibodies.
[6] Heffield W P, Marques J A, Bhakta V & Smith I J (2000) Thromb Res. 99, 613-21. Modulation of clearance of recombinant serum albumin by either glycosylation or truncation.
[7] Wawrzynczak E J, Cumber A J, Henry R V, Parnell G D & Westwood J H (1992). Biochem Soc Trans 20, 738-43. Structural factors influencing the pharmacokinetics and stability of immunotoxins.
[8] Schellekens H (2002) Nat Rev Drug Discov. 1, 457-62. Bioequivalence and the immunogenicity of biopharmaceuticals.
[9] Felnerova D, Viret I F, Gluck R & Moser C (2004). Curr Opin Biotechnol. 15, 518-29 Liposomes and virosomes as delivery systems for antigens, nucleic acids and drugs.
[10] Worn A, Pluckthun A (2001). J Mol. Biol. 305, 989-1010. Stability engineering of antibody single-chain Fv fragments.
[11] Graham M L (2003). Adv Drug Deliv Rev. 55, 1293-302. Pegaspargase: a review of clinical studies
[12] Robins G W, Scott L T & Keating G M (2005). Drugs. 65, 809-25. Peginterferon-alpha-2a (401(D): a review of its use in the management of patients with chronic hepatitis B.
[13] Yang K, Basu A, Wang M et al (2003). Protein Eng. 16, 761-70. Tailoring structure-function and pharmacokinetic properties of single-chain Fv proteins by site-specific PEGylation.
[14] Mosier N M (2003). WO03099226, ANTIBODY PEG POSITIONAL ISOMERS, COMPOSITIONS COMPRISING SAME, AND USE THEREOF. PHARMACIA CORP
[15] Burg J & Engel A (2002). WO0249673 ERYTHROPOIETIN CONJUGATES. HOFFMANN LA ROCHE
[16] Avrarnis V I & Cohen L (1999) WO9939732. PHARMACEUTICAL COMPOSITIONS COMPRISING PEG-ASPARAGINASE FOR THE TREATMENT OF HIV INFECTIONS
[17] van Vlasselaer P & Roberts M J (2004). WO2004076474. POLYETHYLENE GLYCOL MODIFIED INTERFERON COMPOSITIONS AND METHODS OF USE THEREOF. Intermune Inc.
[18] Gregoriadis G, Fernandes A, Mital M & McCormack B (2000). Cell Mol. Life Sci 57 1964-1969. Polysialic acids: potential in improving the stability and pharmacokinetics of proteins and other therapeutics.
[19] Srroda K, Rydlewski J, Langner M et al. (2005). Cell Mol Biol Lett. 10, 37-47. Repeated injections of PEG-PE liposomes generate anti-PEG antibodies.
[20] Steenbergen S M, Vimr E R (2003). J Biol. Chem. 278, 15349-59. Functional relationships of the sialyltransferases involved in expression of the polysialic acid capsules of Escherichia coli K1 and K92 and Neisseria meningitidis groups B or C.
[21] Gregoriadis G, Jain S, Papaioannou I, Laing P (2005). Int J. Pharm. 300, 125-30. Improving the therapeutic efficacy of peptides and proteins: a role for polysialic acids.
[22] Durbec P, Cremer H (2001). Mol. Neurobiol. 24, 53-64. Revisiting the function of PSA-NCAM in the nervous system
[23] Muhlenhoff M, Eckhardt M, Gerardy-Schahn R (1998). Curr Opin Struct Biol. 8, 558-64. Polysialic acid: three-dimensional structure, biosynthesis and function.
[24] Gregoriadis G, McCormack B, Wang Z, Lifely R (1993) FEBS Lett. 315; 271-6. Polysialic acids: potential in drug delivery
[25] Jain S, Hreczuk-Hirst D H, McCormack B et al. (2003). Biochim Biophys Acta. 1622, 42-9. Polysialylated insulin: synthesis, characterization and biological activity in vivo
[26] Fernandes A I, Gregoriadis G (2001). Int J. Pharm. 217, 215-24. The effect of polysialylation on the immunogenicity and antigenicity of asparaginase: implication in its pharmacokinetics
[27] Fernandes A I, Gregoriadis G (1996) Biochim Biophys Acta. 1293, 90-6. Synthesis, characterization and properties of sialylated catalase
[28] Constantinou A, Epenetos A A & Deonarain M P (2005). In preparation for J. Biol. Chem.
[29] Constantinou A, Epenetos A A & Deonarain M P (2005). In preparation for Bioconj. Chem.
[30] Hreczuck-Hirst D & Jain S (2005). WO2005016973 POLYSIALIC ACID DERIVATIVES. Lipoxen Technologies
[31] Jain S & Laing P (2005). WO2005016974 SIALIC ACID DERIVATIVES FOR PROTEIN DERIVATISATION AND CONJUGATION. Lipoxen Technologies
[32] Adams G P, Shaller C C, Chappell L L, Wu C, Horak E M, Simmons H H, Litwin S, Marks J D, Weiner L M & Brechbiel M W (2000) Nucl Med. Biol. 27, 339-46. Delivery of the alpha-emitting radioisotope bismuth-213 to solid tumors via single-chain Fv and diabody molecules.
[33] Lappi D A, Matsunami R, Martineau D, Baird A (1993). Anal Biochem. 212, 446-51. Reducing the heterogeneity of chemically conjugated targeted toxins: homogeneous basic FGF-saporin.
[34] Inoue S & Inoue Y (2001). J Biol. Chem. 276, 31863-70. Developmental profile of neural cell adhesion molecule glycoforms with a varying degree of polymerization of polysialic acid chains.
[35] James W M, Emerick M C & Agnew W S (1989). Biochemistry 28, 6001-9. Affinity purification of the voltage-sensitive sodium channel from electroplax with resins selective for sialic acid.
[36] Yabe U, Sato C, Matsuda T & Kitajima K (2003). J Biol. Chem. 278, 13875-80. Polysialic acid in human milk. CD36 is a new member of mammalian polysialic acid-containing glycoprotein.
[37] Close B E & Colley K J (1998). J Biol Chem. 273, 34586-93. In vivo autopolysialylation and localization of the polysialyltransferases PST and STX.
[38] Walsh F S & Doherty F (1997). Annu Rev. Cell Dev. Biol. 13, 425-56. Neural cell adhesion molecules of the immunoglobulin superfamily: Role in Axon Growth and Guidance
[39] Mendiratta S S, Sekulic N, Lavie A et al. (2005). J Biol. Chem. 280, 32340-8. Specific amino acids in the first fibronectin type III repeat of the neural cell adhesion molecule play a role in its recognition and polysialylation by the polysialyltransferase ST8Sia IV/PST
[40] Nelson R, Bates P A & Rutishauser U (1995). J. Biol. Chem. 270, 17171-17179. Protein determinants for specific polysialylation of the neural cell adhesion molecule.
[41] Johnson C P, Fujimoto I, Rutishauser U & Leckband D E (2005). J Biol. Chem. 280, 137-45. Direct evidence that neural cell adhesion molecule (NCAM) polysialylation increases intermembrane repulsion and abrogates adhesion.
[42] Cremer H, Lange R, Christoph A, Plomann M, Vopper G, Roes J, Brown R, Baldwin S, Kraemer P, Scheff S, et al. (1994) Nature 367, 455-9. Inactivation of the N-CAM gene in mice results in size reduction of the olfactory bulb and deficits in spatial learning.
[43] Seidenfaden R, Krauter A, Schertzinger F, Gerardy-Schahn R & Hildebrandt H (2003). Mol Cell Biol. 23, 5908-18. Polysialic acid directs tumor cell growth by controlling heterophilic neural cell adhesion molecule interactions.
[44] Close B E & Mendiratta S S, Geiger K M et al. (2003). J Biol Chem. 278, 30796-805. The minimal structural domains required for neural cell adhesion molecule polysialylation by PST/ST8Sia IV and STX/ST8Sia II
[45] Kuby (2000). Immunology, 4th Ed. W. H. Freeman.
[46] Hoogenboom H R (2005). Nat Biotechnol. 23, 1105-16. Selecting and screening recombinant antibody libraries
[47] Carter, P (2001) Nat Rev Cancer 1, 118-29. Improving the efficacy of antibody-based cancer therapies.
[48] Wu A M & Senter P D (2005). Nat Biotechnol. 23, 1137-46. Arming antibodies: prospects and challenges for immunoconjugates.
[49] Maynard J A, Maassen C B, Leppla S H, Brasky K, Patterson J L, Iverson B L & Georgiou G. (2002). Nat Biotechnol. 20, 597-601. Protection against anthrax toxin by recombinant antibody fragments correlates with antigen affinity.
[50] Jain R K (1990). Cancer Res. 50 (3 Suppl) 814s-819s. Physiological barriers to delivery of monoclonal antibodies and other macromolecules in tumors.
[51] Little M, Kyprianov S M, LeGall F & Moldenhauer G (2000). Immunol Today 21, 364-70. Of mice and men: hybridoma and recombinant antibodies.
[52] Verhaar M J et al. (1995). Int J Cancer 61, 497-501. A single chain Fv derived from a filamentous phage library has distinct tumor targeting advantages over one derived from a hybridoma.
[53] Epenetos A A, Snook D, Durbin H, Johnson P M, Taylor-Papadimitriou (1986). Cancer Res. 46, 3183-91. Limitations of radiolabeled monoclonal antibodies for localization of human neoplasms.
[54] Gangopadhyay, A et al. (1996). Nucl. Med. Biol. 23, 257-61. Modification of antibody isoelectric point affects biodistribution of 111-indium-labeled antibody.
[55] Deonarain M P & Epenetos A A (1998) Br. J. Cancer. 77, 537-46. Design, characterization and anti-tumour cytotoxicity of a panel of recombinant, mammalian ribonuclease-based immunotoxins.
[56] Linardou, H. et al. (2000) Int. J. Cancer 86, 561-569. A recombinant cytotoxic chimera based on mammalian deoxyribonuclease-I
[57] Dolmans D E, Fukumura D, Jain R K (2003). Nature Rev. Cancer 3, 380-386. Photodynamic therapy for cancer.
[58] Ancey C, Kuster A, Haan S, Hermann A, Heinrich P C & Muller-Newen G (2003) J. Biol. Chem. 278, 16968-72. A fusion protein of the gp130 and interleukin-6Ralpha ligand-binding domains acts as a potent interleukin-6 inhibitor.
[59] Borsi L, Balza E, Carnemolla B, Sassi F, Castellani P, Berndt A, Kosmehl H, Biro A, Sin A, Orecchia P, Grassi J, Neri D & Zardi L (2003) Blood. 102, 4384-92. Selective targeted delivery of TNFalpha to tumor blood vessels.
[60] Korner M, Waser B & Reubi J C (2005) Int J Cancer 115, 734-41. Neuropeptide Y receptors in renal cell carcinomas and nephroblastomas.
[61] Koide A, Bailey C W, Huang X & Koide S (1998) J Mol Biol. 284, 1141-51. The fibronectin type III domain as a scaffold for novel binding proteins.
[62] Holt L J, Herring C, Jespers L S, Woolven B P & Tomlinson I M (2003) Trends Biotechnol. 21, 484-90. Domain antibodies: proteins for therapy.
[63] Schlehuber S & Skerra A (2001) Biol Chem. 382, 1335-42. Duocalins: engineered ligand-binding proteins with dual specificity derived from the lipocalin fold.
[64] Binz H K, Amstutz P, Kohl A, Stumpp M T, Briand C, Forrer P, Grutter M G & Pluckthun A (2004) Nat Biotechnol. 22, 575-82. High-affinity binders selected from designed ankyrin repeat protein libraries.
[65] Adams G P & Weiner L M (2005). Nat Biotechnol. 23, 1147-57. Monoclonal antibody therapy of cancer.
[66] Holliger P & Hudson P J (2005). Nat Biotechnol. 23, 1126-36. Engineered antibody fragments and the rise of single domains.
[67] Sinclair A M, Elliott S (2005). J Pharm Sci. 94, 1626-35. Glycoengineering: the effect of glycosylation on the properties of therapeutic proteins.
[68] Elliott S, Lorenzini T, Asher S, Aoki K, Brankow D, Buck L, Busse L, Chang D, Fuller J, Grant J, Hernday N, Hokum M, Hu S, Knudten A, Levin N, Komorowski R, Martin F, Navarro R, Osslund T, Rogers G, Rogers N, Trail G & Egrie J (2003). Nat Biotechnol. 21, 414-21 Enhancement of therapeutic protein in vivo activities through glycoengineering.
[69] Park L S (1994) U.S. Pat. No. 5,298,395. Hyperglycosylated cytokine conjugates. Immunex Corp
[70] Elliott S, Chang D, Delorme E, Eris T, Lorenzini T (2004). J Biol Chem. 279, 16854-62. Structural requirements for additional N-linked carbohydrate on recombinant human erythropoietin.
[71] Narazaki M & Tosato G (2005). J Natl Cancer Inst. 97, 705-7. Targeting coagulation to the tumor microvasculature: perspectives and therapeutic implications from preclinical studies.
[72] Baca M & Wells J (2003) EP1325932. Anti-VEGF antibodies.
[73] Adair J R & Singh A D (1999) U.S. Pat. No. 5,994,510. Recombinant antibodies specific for TNF alpha
[74] Braun J, Sieper J (2003). Expert Opin Biol Ther. 3, 141-68. Overview of the use of the anti-TNF agent infliximab in chronic inflammatory diseases.
[75] Changgeng R & Qingwei Y E G (2004). EP1382617 Monoclonal antibodies recognizing human platelet GPIIIa and GPIIb/GPIIIa and use thereof in anti-thrombotic therapy
[76] Waruk J L M & Berry J D (2005). WO2005097822. Identification of the precise amino acid sequence recognized by the potent neutralizing human anti-HIV monoclonal antibody IgG1B12.
[77] Mohit T & Zaki M (2004). WO2004071404. use of IL-6 antagonists in combination with steroids to enhance apoptosis.
[78] Brown D R & Smidt J J (1998). U.S. Pat. No. 5,807,741 Neutralizing monoclonal antibody against botulinum neurotoxin serotype F
[79] Kang A S & Wang F (2005). WO2005081749. Neutralizing human antibodies to anthrax toxin.
[80] Cox G N & McDermott M J (1995) WO9532003. Modified insulin-like growth factors.
[81] Burg J & Engel A (2002). WO0249673. Erythropoietin conjugates.
[82] Deshpande R V & Tsai M (2004). WO2004046306. Fully human antibody Fab fragments with human interferon-gamma neutralizing activity.
[83] Kosten T & Owens S M (2005). Pharmacol Ther. 108, 76-85. Immunotherapy for the treatment of drug abuse.
[84] Bloom J D, Meyer M M, Meinhold P, Otey C R, MacMillan D & Arnold F H (2005). Curr Opin Struct Biol. 15, 447-52. Evolving strategies for enzyme engineering.
[85] Fernandez-Gacio A, Uguen M, Fastrez J (2003). Trends Biotechnol. 21, 408-14. Phage display as a tool for the directed evolution of enzymes.
[86] Lin M Z, Teitell M A, Schiller G J (2005). Clin Cancer Res. 11, 129-38. The evolution of antibodies into versatile tumor-targeting agents.
[87] Angata K, Chan D, Thibault J & Fukuda M (2004). J Biol Chem. 279, 25883-90. Molecular dissection of the ST8Sia IV polysialyltransferase. Distinct domains are required for neural cell adhesion molecule recognition and polysialylation.
[88] Angata K, Nakayama J, Fredette B, Chong K, Ranscht B & Fukuda M (1997). J Biol Chem. 272, 7182-90. Human STX polysialyltransferase forms the embryonic form of the neural cell adhesion molecule. Tissue-specific expression, neurite outgrowth, and chromosomal localization in comparison with another polysialyltransferase, PST.
[89] Franceschini & Fukuda (2001) Glycobiology 11, 231-39. Polysialyltransferase ST8Sia II (STX) polysialylates all of the major isoforms of NCAM and facilitates neurite outgrowth
[90] Annunziato P W, Wright L F, Vann W F & Silver R P (1995). J Bacteriol. 177, 312-9. Nucleotide sequence and genetic analysis of the neuD and neuB genes in region 2 of the polysialic acid gene cluster of Escherichia coli K1.
[91] Hammerschmidt S, Birkholz C, Zahringer U, Robertson B D, van Putten J, Ebeling O & Frosch M (1994). Mol Microbiol. 11, 885-96. Contribution of genes from the capsule gene complex (cps) to lipooligosaccharide biosynthesis and serum resistance in Neisseria meningitidis.
[92] C Sato, K Kitajima, I Tazawa, Y Inoue, S Inoue & F A Troy (1993). J. Biol. Chem. 268, 23675-23684. Structural diversity in the alpha 2->8-linked polysialic acid chains in salmonid fish egg glycoproteins. Occurrence of poly(Neu5Ac), poly(Neu5Gc), poly(Neu5Ac, Neu5Gc), poly(KDN), and their partially acetylated forms.
[93] Sambrook J & Russel D W (1991). Molecular Cloning: A Laboratory Manual. Cold Spring Harbour.

1. A method of polysialylation comprising the steps of:
(i) providing a molecule comprising a first protein or domain thereof associated with a second protein or domain thereof containing a natural polysialylation site;
(ii) exposing the molecule of step (i) to a polysialyltransferase enzyme so as to produce a naturally polysialylated molecule wherein the polysialylation is a sugar chain N-linked onto an asparagine amino acid.

2. A method as claimed in claim 1 wherein the first protein or domain thereof is associated with the second protein or domain thereof containing a natural polysialylation site by either conjugation or fusion.

3. A method as claimed in claim 1 wherein the molecule provided in step (i) is provided by expression of the molecule in a host cell.

4. A method as claimed in claim 1 wherein step (ii) occurs in the host cell by the cell containing a polysialyltransferase enzyme.

5. A method as claimed in claim 1 wherein an unmodified first protein or domain thereof is modified to include a domain comprising a natural polysialylation site.

6. A method as claimed in claim 5 wherein the second domain comprising a natural polysialylation site contains at least one glycosylation motif having the amino acid sequence Asn-X-Thr/Ser.

7. A method as claimed in claim 1 wherein the first protein or domain thereof is an antibody, ligand or enzyme.

8. A method as claimed in claim 7 wherein the first protein is an antibody.

9. A method as claimed in claim 7 wherein the first protein is an scFv.

10. A method as claimed in claim 1 wherein the second protein or domain thereof containing a natural polysialylation site is derived from a protein selected from the list of: Neural Cell Adhesion Molecule (NCAM); alpha sub-unit of voltage gated sodium channel, CD36 scavenger receptor, ST8Ssia IV/PST polysialyltransferase (PST); STSSia II/STX polysialyltransferase (STX); capsid of E. coli strain KI; capsid of Neisseria meningitides group B; fish egg glycoprotein and modified forms thereof.

11. A method as claimed in claim 10 wherein the second protein or domain thereof containing a natural polysialylation site is derived from NCAM and modified forms thereof.

12. A method as claimed in claim 11 wherein the second protein or domain thereof containing a natural polysialylation site is the fifth immunoglobulin domain (Ig5 domain) of NCAM.

13. A method as claimed in claim 12 also comprising the first type-III fibronectin-like domain (FN_{III}-1) of NCAM.

14. A method as claimed in claim 12 comprising a plurality of Ig5 domains.

15. A method as claimed in claim 13 comprising a plurality of Ig5 and a plurality of FN_{III}-1 domains.

16. A method as claimed in claim 1 wherein conjugated first protein or a modified polysialylated protein possesses altered polysialylation levels, size and/or mass; immunogenicity, blood circulation half-life and/or proteolytic stability, wherein the altered state may be increased or decreased in comparison to the wildtype protein.

17. A method as claimed in claim 1 wherein the molecule of step (i) also comprises one or more additional sequences selected from the list of: secretion signal sequences; membrane anchoring sequences (e.g. transmembrane domains or GPI-anchors); protease cleavage sites, domains for aiding detection and/or purification (e.g. hexahistidine sequence).

18. A method as claimed in claim 2 wherein the expressed fusion protein is optionally cleaved to remove at least one non-polysialylated domain.

19. A method as claimed in claim 1 wherein the molecule of step (i) has the amino acid sequence of FIG. 9.

20. A polysialylated molecule obtainable by the method as described in claim 1.

21. A nucleic acid having a nucleotide sequence encoding the polysialylated molecule as defined in claim 20.

22. A nucleic acid as claimed in claim 21 having the nucleotide sequence of FIG. 9.

23. An expression vector containing a nucleotide sequence encoding the polysialylated molecule as defined in claim 20.

24. An expression vector as claimed in claim 23 wherein the nucleotide sequence encoding the polysialylated molecule is that of FIG. 9.

25. A host cell producing a polysialylated molecule as defined in claim 20 resulting from expression of the nucleotide sequence encoding the polysialylated molecule.

26. A host cell as claimed in claim 25 wherein the nucleotide sequence encoding the polysialylated molecule is that of FIG. 9.

27. A composition comprising the polysialylated molecule as defined in claim 20 and a pharmaceutically acceptable carrier, excipient and/or diluent.

28. A polysialylated molecule as defined in claim 20 for use in the treatment of disease.

29. Use of a polysialylated molecule as defined in claim 20 in the manufacture of a medicament for the treatment and/or diagnosis and/or prevention of solid cancer (e.g. breast, prostate, lung, renal, colorectal), disseminated cancers (e.g. lymphomas and leukaemias), infectious diseases (e.g. malaria, leishmanaisis, meningitis, botulinum poisoning, E. coli, influenza, HIV, hepatitis), narcotics poisoning (e.g. cocaine) and cardiovascular diseases (blood clots, heart disease).

30. Use of a polysialylated molecule as defined in claim 20 in a screening assay.

31. A use as claimed in claim 30 wherein the screening assay comprises identifying antibodies, antibody fragments or antibody derivatives that are able to bind a target molecule.

32.-39. (canceled)

40. A composition as defined in claim 27 for use in the treatment of disease.
